(19) United States
(12) Patent Application Publication
(10) Pub. No.: US 2024/0271037 A1
(43) Pub. Date: Aug. 15, 2024

(54) HIGH PERFORMANCE AQUEOUS HALIDE PEROVSKITE NANOCRYSTALS

(71) Applicant: Nanyang Technological University, Singapore (SG)

(72) Inventors: Tze Chien SUM, Singapore (SG); Huajun He, Singapore (SG)

(21) Appl. No.: 18/567,543

(22) PCT Filed: Jul. 6, 2022
(86) PCT No.: PCT/SG2022/050469

§ 371 Date: Dec. 6, 2023

(30) Foreign Application Priority Data
Jul. 6, 2021 (SG) 1020210741Q
Feb. 4, 2022 (SG) 10202201079R

(51) Int. Cl. C09K 11/02; A61K 49/00; B82Y 5/00; B82Y 20/00; B82Y 40/00; C09K 11/66; C09K 11/77

(52) U.S. Cl. CPC C09K 11/025; A61K 49/0067; C09K 11/665; C09K 11/7705; B82Y 5/00; B82Y 20/00; B82Y 40/00

(57) ABSTRACT

The present invention relates to a nanocrystal having a core-shell structure, wherein the core comprises a core perovskite structure, and the shell comprises a shell perovskite structure and a compound comprising silicon and oxygen, wherein the shell per-ovskite structure is different from the core perovskite structure and comprises a low-dimensional perovskite structure that is doped 5 with a metal halide comprising a monovalent, divalent or trivalent metal ion. The present invention also relates to a process for preparing the nanocrystal, a substrate comprising the nanocrystal and the use of the nanocrystal.

### CROSS-REFERENCE TO RELATED APPLICATION

This application claims the benefit of both priorities of Singapore Patent Application No. 10202107417Q filed on 6 Jul. 2021 and 10202201079R filed on 4 Feb. 2022, the contents of both being hereby incorporated by reference in its entirety for all purposes.

### TECHNICAL FIELD

The present invention relates to a nanocrystal having a core-shell structure, wherein the core comprises a core perovskite structure, and the shell comprises a shell perovskite structure and a compound comprising silicon and oxygen, wherein the shell perovskite structure is different from the core perovskite structure and comprises a low-dimensional perovskite structure that is doped with a metal halide comprising a monovalent, divalent or trivalent metal ion. The present invention also relates to a process for preparing the nanocrystal, a substrate comprising the nanocrystal and the use of the nanocrystal.

### BACKGROUND ART

Halide perovskite nanocrystals (HPNCs) with general formula ABX₃, where A=CH₃NH₃⁺, CH(NH₂)₂⁺, Cs⁺, Rb⁺; B=Pb²⁺, Sn²⁺, Ge²⁺; X═I⁻, Br⁻, Cl⁻, F⁻ are highly promising emergent nanomaterials with outstanding optoelectronic properties such as high carrier mobilities, long carrier diffusion lengths. defect tolerance, tunable emission wavelengths, large linear absorption and multi-photon absorption (MPA) cross-sections and near-unity photoluminescence quantum yield (PLQY). Recently, functional ion doping or substitution with halogens, transition metals (Mn and Fe) and rare earths (Er and Yb) have not only enabled low-lead or lead-free HPNCs with reduced toxicity, but have also facilitated further tailoring of their optical and electromagnetic properties for applications in sensing, spintronics, and quantum cutting, in particular in light-emitting diodes (LEDs), solar cells, photodetectors, and lasers.

Nevertheless, all HPNCs (with or without doping) suffer from an inherent major weakness of poor ambient stability such as water, oxygen, heat or irradiation. In particular, water degradation severely limits their practical applications. Even trace amounts of water (H₂O) can drastically affect their performance. Most stability studies of HPNCs dispersed in water have only been performed using the green-emitting MAPbBr₃ (MA=CH₃NH₃⁺) and CsPbBr₃. Related studies on their chloride (blue emission) and iodide (red or infrared emission) counterparts are few and far between due to their much poorer water stability likely from their lower defect tolerance and ion migration issues. Research in functional ion-doped or substituted perovskite nanocrystals is still nascent with most studies focused mainly on basic optical characterization.

Nonetheless, the adverse effects of water on these perovskite nanocrystals are still expected to be present, as: (i) water molecules can destroy the nanocrystal structure, and/or (ii) coupling of the high-frequency stretching vibration of O—H in water molecules with most B-site doped ions, result in non-radiative decay and fluorescence quenching. Presently, it is extremely challenging to prepare colloidal HPNCs that emit across the full colour spectrum that also remain stable in water even for one day. Accordingly, existing approaches to tune the broad emission spectrum of perovskite nanocrystals relies on the use of organic halides with high solubility in organic systems, such as haloalkane solvents used with a strong nucleophile, long alkyl-based oleylammonium iodide and aryl-based aniline hydoroiodide, to alter the properties of the HPNCs.

Despite possessing the largest MPA cross-sections, the instability of the HPNCs in water has also severely curtailed the potential of colloidal HPNCs as a fluorophore for multi-photon bioimaging applications. Ultra-bright low-toxicity multicolour HPNCs with greatly enhanced water, light and chromatic stabilities will extend the boundaries of lighting, flat panel display and biological imaging technologies.

There is therefore a need for development of a nanocrystal that overcomes or at least ameliorates, one or more of the disadvantages described above.

### SUMMARY

In an aspect, there is provided a nanocrystal having a core-shell structure, wherein the core of the core-shell structure is at least partially encapsulated by the shell of the core-shell structure, wherein:
the core of the core-shell structure comprises a core perovskite structure having a formula ABX₃, wherein:
   A is selected from the group consisting of at least one ion of one or more group 1 elements of the Periodic Table of Elements, an organic cation having a structure of R¹—(NHₓ)_{y}⁺ wherein R¹ is CH or alkyl, x is 2 or 3 and y is 1 or 2, as valency allows, and any mixture thereof:
   B is at least one ion of one or more group 14 elements of the Periodic Table of Elements; and
   X is a halide ion or any mixture thereof, and

the shell of the core-shell structure comprises a shell perovskite structure and a compound comprising silicon and oxygen, wherein the shell perovskite structure is different from the core perovskite structure and comprises a low-dimensional perovskite structure that is doped with a metal halide comprising a monovalent, divalent or trivalent metal ion.

Advantageously, the nanocrystal as defined above may have high brightness and may emit across the full colour spectrum depending on the metal halide used to dope the low-dimensional perovskite structure.

The nanocrystal may advantageously have high dispersibility in water and may possess excellent water stability. Such stability and dispersibility may be unprecedented. For example, in water, the green and blue emitting nanocrystals may possess a photoluminescence quantum yield (PLQY) of ≥80% and ≥35%, respectively, for more than 7720 hours and 7000 hours, respectively. For the red-emitting HPNCs, the PLQY in water may reach about 30% for about 750 hours, which may be a significant improvement compared to conventional red-emitting unprotected iodide perovskite nanocrystals, whose fluorescence may rapidly quench when in contact with moisture. Further advantageously, the low-dimensional perovskite structures on the surface of the core perovskite structure may confer excellent passivation, protection, and biocompatibility to the nanocrystal in conjunction with the compound comprising silicon and oxygen, which may at least partially encapsulate the nanocrystal, thereby improving its stability.

Advantageously, the nanocrystals may possess high multiphoton action cross-sections in water that may be 4 to 5 orders of magnitude larger than that of the most advanced, specially designed organic molecules in organic solutions.

Further advantageously, the nanocrystals may have low biological toxicity.

Advantageously, the presence of the metal halide and the compound comprising silicon and oxygen may synergistically facilitate high performance, high emission across the full colour spectrum at high PLQY, as well as high stability and dispersibility in water, and low toxicity.

In another aspect, there is also provided a process of preparing the nanocrystal as defined above, comprising a step of simultaneously mixing in a mixing solvent, a core perovskite structure having a formula ABX₃, a metal halide comprising a monovalent, divalent or trivalent metal ion and a precursor compound comprising silicon and oxygen, wherein:
A is selected from the group consisting of at least one ion of one or more group 1 elements of the Periodic Table of Elements, an organic cation having a structure of R¹—(NHₓ)_{y}⁺ wherein R¹ is CH or alkyl, x is 2 or 3 and y is 1 or 2, as valency allows, and any mixture thereof:
B is at least one ion of one or more group 14 elements of the Periodic Table of Elements; and
X is a halide ion.

Advantageously, the method may achieve ion doping or substitution of both the core and shell of the core-shell structure, wherein the shell comprises the shell perovskite structure and the compound comprising silicon and oxygen. The ion doping or substitution may basically occur in the perovskite structure. The synergistic implementation of these two processes may circumvent introduction of unnecessary surface defects on the nanocrystals, which may be common in typical multi-step syntheses. This may result in better surface passivation of the nanocrystals, thus ensuring that the nanocrystals may maintain high performance while improving water stability.

The method may further advantageously be performed at low temperatures and may be a simple chemical synthesis under atmospheric conditions, which may facilitate large scale, cost-efficient and time-efficient preparation of the nanocrystals. This may be contrary to conventional perovskite nanocrystal synthesis, which may require high temperature or inert atmosphere preparation. Further advantageously, the method may not only be highly versatile and cost-effective, but may also result in the formation of perovskite nanocrystals that may emit across the full colour spectrum.

In an example, prior to the mixing step, the process may further comprise the step of dissolving the metal halide in a polar solvent comprising an alcohol, a fatty acid, a fatty amine, and an amine having a structure N(R²)₃, wherein R² may be independently hydrogen or alkyl.

Advantageously, the polar solvent may facilitate the complete dissolution of the metal halide at high concentrations and thereby enable post-treatment of the core perovskite structure for doping or substitution with the metal halide. The polar solvent may further be miscible with low-polarity solvents that may be present in the mixing solvent to aid the dispersion of the core perovskite structure and/or the compound comprising silicon and oxygen. This may facilitate the mixing solvent to be a good homogeneous system for ion-doping and shelling process.

The method as defined above may be contrary to conventional wisdom, as non-polar or low polarity post treatment solutions may be typically used in conventional synthesis of perovskite nanocrystals to minimize potential damage to the perovskite nanocrystal structures. The use of a suitable polar solvent may adjust the hydrophilicity of the synthesized nanocrystal, so that the nanocrystals may be highly dispersible in water. Advantageously, the method as defined above may not only circumvent the issue of poor solubility of most metal halides in low polarity solvents. but may also enable doping or substitution with functional metal ions. Importantly, the method may enable self-repairing of the perovskite nanocrystals, thus ensuring high PLQY.

In another aspect, there is also provided a substrate comprising the nanocrystal as defined above, wherein the substrate may be selected from the group consisting of an aqueous solution, film, microcrystal, or bulk single crystal.

In another aspect, there is also provided the use of the nanocrystal as defined above or the substrate as defined above in LEDs, multi-photon imaging, full-colour displays, lasers, bioimaging, optoelectronics, spintronic devices, solar cells, memristors or as radiation detectors.

Advantageously, the combination of high performance, high stability and high dispersibility in water of the nanocrystals may be useful in the development of HPNCs for practical applications.

### Definitions

The following words and terms used herein shall have the meaning indicated:

The word “core-shell” refers to a structure in which the core is at least partially encapsulated by the shell, where the core and shell are two different materials.

The word “low-dimensional perovskite” refers to molecular-level or structure-level low-dimensional perovskite structures which are either perovskite structures a) made of the [BX₆]⁴⁻ octahedra that exist in a two-dimensional, one-dimensional or zero-dimensional form, as opposed to a three-dimensional form. They possess the dimension to one or several molecular units in at least one direction; or b) having a morphology of a nanoplatelet, nanosheet, nanowire, nanorods, nanocrystals or nanoclusters.

The word “doped” and “substituted” with respect to the metal halide in the nanocrystal as defined herein, may be used interchangeably, and refers to the intentional introduction of an impurity into the intrinsic perovskite for the purpose of modulating its electrical, optical and structural properties. The term “doping” and “substituting” should be construed accordingly.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### BRIEF DESCRIPTION OF DRAWINGS

The accompanying drawings illustrate a disclosed embodiment and serve to explain the principles of the disclosed embodiment. It is to be understood, however, that the drawings are designed for purposes of illustration only, and not as a definition of the limits of the invention.

FIG. 1 refers to a synthesis schematic of highly stable full colour emitting aqueous halide perovskite nanocrystals (HPNCs). The prepared CsPbBr₃ nanocrystals were subjected to wet chemical treatments of metal halide and tetramethylorthosilicate (TMOS) at the same time. This not only achieved the adjustment of full-colour emission due to an ion doping or substitution process, but also resulted in the formation of a hybridized shell assembled from low-dimensional perovskites and silicon-oxygen compounds, thus affording excellent surface passivation and protection, high dispersibility and low toxicity.

FIG. 2 refers to a set of images showing the basic characterization of the template CsPbBr₃ NCs. (a) are high resolution transmission electron microscopy (HRTEM) images of the as-synthesized CsPbBr₃ NCs viewed along the zone axis. Scale bar, 10 nm. Inset is the fast Fourier transform (FFT) patterns from which the lattice spacings were derived from and indexed according to the CsPbBr₃, Pnma crystal structure. Scale bar in FFT, 2 1/nm: (b) is the absorbance and photoluminescence (PL) spectra of the as-synthesized CsPbBr, NCs in toluene: (c) is the two-dimensional pseudo-colour transient absorption (TA) plot of the of the as-synthesized CsPbBr₃ NCs in toluene, where “+” denotes that the region is in a positive signal while “−” denotes that the region is in a negative signal. (d) is the two-dimensional contour image of time-resolved PL (TRPL) decay of CsPbBr₃ NCs in toluene, where “+” denotes that the region is in a positive signal while “−” denotes that the region is in a negative signal; and (e) is the CIE chromaticity spectrum of the as-synthesized CsPbBr₃ NCs in toluene.

FIG. 3 refers to a photographic image showing that the mixed solvent system of alcohol/O Ac/O Am/ammonia facilitates the formation of clear solutions of most metal halide salts, with a concentration of 0.02 M to 0.4 M.

FIG. 4 refers to a set of graphs showing the basic photoluminescence (PL) properties of aqueous HPNCs. (a) is the absorbance and PL spectra of HPNCs treated with TMOS and representative metal halides MnCl₂, YCl₃, ZnCl₂, InBr₃, NiI₂, InI₃, and ZnI₂, measured in water; (b) are photographic images of HPNCs treated with TMOS and representative metal halides MnCl₂, YCl₃, ZnCl₂, InBr₃, NiI₂, InI₃ and ZnI₂, that are dispersed in water and under bright field or photoluminescence conditions, showing the full range of colours from magenta to dark red; (c) is a CIE chromaticity spectrum of the HPNCs treated with representative metal halides and TMOS, measured in water, displaying coverage of the full-colour emission spectrum; (d) refers to two-dimensional pseudo-colour TA plots of HPNCs treated with representative metal halides (d1) YCl₃, (d2) InBr₃ and (d3) NiI₂ and TMOS, measured in water, where “+” denotes that the region is in a positive signal while “−” denotes that the region is in a negative signal.; (e) is a two-dimensional contour image of TRPL decay of HPNCs treated with representative metal halides (e1) YCl₃, (e2) InBr₃ and (e3) NiI₂ and TMOS, measured in water, where “+” denotes that the region is in a positive signal while “−” denotes that the region is in a negative signal.; (f) is a comparison of the TRPL between the pristine CsPbBr₃ NCs in toluene and HPNCs treated with representative metal halides (YCl₃, InBr₃ and NiI₂) and TMOS in water: and (g) shows the emission full width at half-maximum (FWHM) of the HPNCs treated with representative metal halides and TMOS, measured in water.

FIG. 5 refers to a set of graphs showing the absorbance and PL spectra of the template CsPbBr₃ dispersed in hexane, HPNCs treated with PbBr₂ and TMOS, dispersed in hexane and HPNCs treated with PbBr₂ and TMOS, dispersed in water.

FIG. 6 refers to a set of graphs showing the detailed TEM measurements of the HPNC sample treated with InBr₃-0.5 and TMOS. (a) is a graph showing the size distribution of the overall nanoparticle of the HPNC sample treated with InBr₃-0.5 and TMOS; (b) is a graph showing the size distribution of the core of the HPNC sample treated with InBr₃-0.5 and TMOS; (c) is the energy dispersive X-ray (EDX) spectrum of a representative single nanoparticle where In and Si signals are just above the noise level due to the low concentration of these two elements and the limited total electron dose used to mitigate electron-beam induced degradation. (d-f) are overlay scanning transmission electron microscopic (STEM) and EDX mapping of the element (d) Cs, (e) Pb, and (f) Br. Pb is detected to be mainly concentrated at the core area and less distributed at the shell layer; (g) is an STEM and EDX mapping of the element Cs, Pb, and Br in the core and shell area; and (h) is a table showing the EDX atomic % calculated using the Cliff-Lorimer method. The k factors were taken from the k-factor library in the JEOL software. Scale bars, 10 nm.

FIG. 7 refers to a set of images showing the structure and composition characterization of representative aqueous HPNCs. (a) shows the powder X-ray diffraction (PXRD) patterns of the pristine CsPbBr₃ NCs, and HPNCs treated with representative metal halides (PbBr₂-0.2, InBr₃-0.5, MnCl₂-1.0 (½) and NiI₂-0.25) and TMOS. BW: before water treatment, AW: after water treatment; (b) is set of images showing (b0) a transmission electron microscopy (TEM) analysis of HPNCs treated with InBr₃-0.5 and TMOS, and high resolution TEM (HRTEM) images of the core (b1) and shell (b2). The associated fast Fourier transform (FFT) patterns viewed in the [211] and [311] zone axis directions for the shell and the core, are also shown. The core image contains overlapping information from the core and the shell, where the most intense reflections were from the core (indicated in dotted circles) and indexed according to the CsPbBr₃ Pnma crystal structure. The shell FFT pattern was indexed according to the Cs₄PbBr₆ trigonal R-3c space group. STEM images were recorded prior the acquisition of EDX dataset and EDX spectroscopic elemental maps. Scale bars in the HRTEM. STEM and EDX images, 10 nm. Scale bars in the FFT patterns. 1 1/nm; (c) is an X-ray photoelectron spectroscopy (XPS) full scan analysis of the pristine CsPbBr₃ NCs, and HPNCs treated with representative metal halides (PbBr₂-0.2, InBr₃-0.5, MnCl₂-1.0 (½) and NiI₂-0.25) and TMOS; and (d) are Fourier-transform infrared (FTIR) spectra of the pristine CsPbBr₃ NCs, and HPNCs treated with representative metal halides (PbBr₂-0.2, InBr₃-0.5, MnCl₂-1.0 (½) and NiI₂-0.25) and TMOS.

FIG. 8 refers to a set of transmission electron microscope (TEM) micrographs showing (a) CsPbBr₃ nanocrystals post-treated with PbBr₂; and (b) CsPbBr₃ nanocrystals post-treated with PbBr₂ and TMOS.

FIG. 9 refers to a set of graphs showing (a-e) high resolution XPS profiles of (a) Cs 3d, (b) Pb 4f, (c) Br 3d, (d) Si 2p and (e) O 1s in HPNCs treated with PbBr₂-0.2 & TMOS; (f-k) high resolution XPS profiles of (f) Cs 3d, (g) Pb 4f, (h) Br 3d, (i) In 3d, (j) Si 2p and (k) O 1s in the HPNCs treated with InBr₃-0.5 and TMOS; (I-r) high resolution XPS profiles of (I) Cs 3d, (m) Pb 4f, (n) Br 3d, (o) Mn 2p, (p) Cl 2p, (q) Si 2p and (r) O 1s in HPNCs treated with MnCl₂-1.0 (1/2) and TMOS; and (s-y) high resolution XPS profiles of (s) Cs 3d, (t) Pb 4fz, (u) Br 3d, (v) Ni 2p, (w) I 3d, (x) Si 2p and (v) O 1s of HPNCs treated with NiI₂-0.25 and TMOS. A: integral area; S: relative sensitivity factor. All A/S only calculate the two strongest peaks.

FIG. 10 refers to a set of graphs showing the stability characterization of the as-synthesized aqueous HPNCs. (a) is a graph showing dispersion time-dependent PLQY spectra of HPNCs treated with representative metal halides (YCl₃, MnCl₂, InBr₃, and NiI₂) and TMOS, measured in water, (b) is a CIE chromatogram showing colour stability dependence by dispersion time of HPNNCs treated with TMOS and representative metal halides (YCl₃, MnCl₂, InBr₃ and NiI₂), in water.; (c) is a graph showing the performance of the irradiation source (MF-2000W-LED) used in the light stability experiment, which was calibrated to be equivalent to 1 sun irradiation and in the range of 400 nm to 800 nm. By calibrating the MF-2000W-LED, the relative current of Si solar cell under standard AM 1.5 G irradiance as defined by the American Society for Testing and Materials, was the same as the relative current of Si solar cell under experimental irradiance of MF-2000W-LED; (d) is a graph showing continuous irradiation time-dependent PLQY of the HPNCs treated with InBr₃ and TMOS, measured in water; and (e) is the PL and CIE spectral analysis of the HPNCs treated with InBr₃ and TMOS in water after continuous 1 Sun irradiation for 0, 6, 12, 18 and 24 hours.

FIG. 11 refers to a set of images describing the water induced phase transformation in the zero-dimensional perovskite shell. (a-b) are TEM images of the as-synthesized HPNCs treated with InBr₃ and TMOS, dispersed in (a) hexane and (b) water. Scale bar, 20 nm; (c) is the absorbance and PL spectra of HPNCs treated with InBr₃ and TMOS, dispersed in hexane and in water; and (d) is a schematic drawing showing the possible structural evolution of the core-shell perovskite nanocrystals before and after dispersion in water.

FIG. 12 refers to a set of images showing the zero-dimensional CsPbBr₃ magic sized clusters (MSCs) in an aqueous system. (a) is a TEM image of the uniformly dispersed MSCs, Scale bar, 50 nm; and (b) is a TEM image of the self-assembled MSCs, which showed a two-dimensional thin layered structure, Scale bar, 100 nm.

FIG. 13 refers to a set of images showing (a) the absorbance and PL spectra of HPNCs treated with representative metal halides (MnCl₂, YCl₃, InBr₃ and NiI₂) and TMOS in water; and (b) are TEM images of HPNCs post-treated with representative metal halides (MnCl₂, YCl₃, InBr₃ and NiI₂) and TMOS after being dispersed in water, Scale bar 20 nm.

FIG. 14 refers to a set of graphs showing the multiphoton excitation (MPE) performance of the reference sample, commercially available CdSe/ZnS quantum dots (QDs). (a) is an absorbance and PL spectra of the reference sample. Inset: the PL image of the CdSe/ZnS QDs toluene solution; (b) is the CIE chromaticity spectrum of the CdSe/ZnS QDs toluene solution; (c-d) are two-dimensional pseudo-colour TA plots with excitation wavelength at (c) 400 nm (1PE), and (d) 800 nm (2PE), where “+” denotes that the region is in a positive signal while “−” denotes that the region is in a negative signal; (e) is the TA kinetics at the bleaching signal with excitation wavelength of 400 nm, and 800 nm; (f-g) are TA spectra in the region of bleaching signal at the delay time of 2000 ps, with excitation wavelength at (f) 400 nm and (g) 800 nm: (h-i) are graphs showing the |ΔOD| as the function of photon fluence for (h) 400 nm excitation or equivalent photon fluence for multiphoton excitation at (i) 800 nm at the delay time of 2000 ps. The curves are the best fit to Equation 1, which was used to derive the multiphoton absorption cross-sections for comparison with reference values.

FIG. 15 refers to a set of graphs showing the multiphoton absorption and emission performance of representative aqueous HPNCs. (a) is the pump fluence dependent multiphoton excited PL spectra of HPNCs treated with TMOS and metal halides including YCl₃ (1700 nm excitation, 4PA), InBr₃ (2100 nm excitation, 5PA) and NiI₂ (800 nm excitation, 2PA), tested in water; (b) is the pump fluence dependent multiphoton excited PL intensity in logarithmic coordinates, where the slope represents the order of multiphoton absorption involved; (c-e) is the TA kinetics at the bleaching signal of HPNCs treated with TMOS and (c) YCl₃, (d) InBr₃ and (e) NiI₂ at different excitation wavelengths, tested in water; and (f-h) are PL decay traces of HPNCs treated with TMOS and (f) YCl₃, (g) InBr₃ and (h) NiI₂ at different excitation wavelengths, tested in water.

FIG. 16 refers to a set of graphs showing the multiphoton excited PL and TA performance of the pristine CsPbBr₃ NCs, in toluene. (a) is the PL spectra of pristine CsPbBr₃ NCs in toluene with excitation wavelength at 800 nm (2PE), 1200 nm (3PE), 1700 nm (4PE) and 2100 nm (5PE); (b) is the pump fluence dependent multiphoton excited PL intensity in logarithmic coordinates, where the slope represents the order of multiphoton excitation involved. (c) is the PL decay traces of the pristine CsPbBr₃ NCs with excitation wavelength at 800 nm (2PE), 1200 nm (3PE), 1700 nm (4PE), and 2100 nm (5PE); (d-g) are two-dimensional contour images of the TRPL decay of the pristine CsPbBr. NCs in toluene with excitation wavelength at (d) 800 nm, (e) 1200 nm, (f) 1700 nm, and (g) 2100 nm, where “+” denotes that the region is in a positive signal while “−” denotes that the region is in a negative signal; (h-k) are two-dimensional pseudo-colour TA plots with excitation wavelength at (h) 800 nm (2PE), (i) 1200 nm (3PE), (j) 1700 nm (4PE), and (k) 2100 nm (5PE), where “+” denotes that the region is in a positive signal while “−” denotes that the region is in a negative signal; (1) is a graph comparing the TA kinetics at the bleaching signal with excitation wavelength of 400 nm, 800 nm, 1200 nm, 1700 nm and 2100 nm; (m-q) are TA spectra in the region of bleaching signal at the delay time of 1000 ps, with excitation wavelength at (m) 400 nm, (n)800 nm, (o) 1200 nm, (p) 1700 nm and (q) 2100 nm; (r-v) are graphs showing the |ΔOD| as a function of photon fluence for (r) 400 nm excitation or equivalent photon fluence for multiphoton excitation at (s) 800 nm, (t) 1200 nm, (u) 1700 nm and (v) 2100 nm at a delay time of 1000 ps. The curves are the best fit to Equation 1, which was used to derive the multiphoton absorption cross-sections.

FIG. 17 refers to a set of graphs showing the multiphoton excited PL and TA performance of HPNCs treated with YCl₃-1.0 and TMOS (blue-emitting), measured in water. (a) is the PL spectra with excitation wavelength at 800 nm (2PE), and 1200 nm (3PE). (b-d) are two-dimensional contour images of TRPL decay with excitation wavelength at (b) 800 nm, (c) 1200 nm, and (d) 1700 nm, where “+” denotes that the region is in a positive signal while “−” denotes that the region is in a negative signal; (e-g) are two-dimensional pseudo-colour TA plots with excitation wavelengths at (c) 800 nm (2PE), (f) 1200 nm (3PE), and (g) 1700 nm (4PE), where “+” denotes that the region is in a positive signal while “−” denotes that the region is in a negative signal: (h-k) are TA spectra in the region of bleaching signal at the delay time of 1000 ps, with excitation wavelength at (h) 400 nm, (i) 800 nm, (j)1200 nm, and (k)1700 nm, (l-o) are graphs showing |ΔOD| as the function of photon fluence for (1) 400 nm excitation or equivalent photon fluence for multiphoton excitation at (m) 800 nm, (n) 1200 nm and (o) 1700 nm at the delay time of 1000 ps. The curves are the best fit to Equation 1, which was used to derive the multiphoton absorption cross-sections.

FIG. 18 refers to a set of graphs showing the multiphoton excited PL and TA performance of HPNCs treated with InBr₃-0.5 and TMOS (green-emitting), measured in water. (a) is the PL spectra with excitation wavelength at 800 nm (2PE), 1200 nm (3PE), and 1700 nm (4PE). (b-e) are two-dimensional contour image of TRPL decay with excitation wavelength at (b) 800 nm, (c) 1200 nm, (d) 1700 nm and (e) 2100 nm, where “+” denotes that the region is in a positive signal while “−” denotes that the region is in a negative signal; (f-i) are two-dimensional pseudo-colour TA plots with excitation wavelength at (f) 800 nm (2PE), (g) 1200 nm (3PE), (h) 1700 nm (4PE) and (i) 2100 nm (5PE), where “+” denotes that the region is in a positive signal while “−” denotes that the region is in a negative signal; (j-n) are TA spectra in the region of bleaching signal at the delay time of 1000 ps, with excitation wavelength at (j) 400 nm, (k) 800 nm, (I) 1200 nm, (m) 1700 nm and (n) 2100 nm. (o-s) are graphs showing |ΔOD| as the function of photon fluence for (o) 400 nm excitation or equivalent photon fluence for multiphoton excitation at (p) 800 nm, (q) 1200 nm, (r) 1700 nm, and (s) 2100 nm at the delay time of 1000 ps. The curves are the best fit to Equation 1, which was used to derive the multiphoton absorption cross-sections.

FIG. 19 refers to a set of graphs showing multiphoton excited PL and TA performance of HPNCs treated with NiI₂-0.25 and TMOS (red-emitting), measured in water. (a) is a two-dimensional contour image of TRPL decay with excitation wavelength at 800 nm, where “+” denotes that the region is in a positive signal while “−” denotes that the region is in a negative signal; (b-c) are two-dimensional pseudo-colour TA plots with excitation wavelength at (b) 800 nm (2PE), and (c) 1200 nm (3PE), where “+” denotes that the region is in a positive signal while “−” denotes that the region is in a negative signal; (d-f) are TA spectra in the region of bleaching signal at the delay time of 300 ps, with excitation wavelength at (d) 400 nm, (e) 800 nm and (f) 1200 nm; (g-i) are graphs showing |ΔOD| as the function of photon fluence for (g) 400 nm excitation or equivalent photon fluence for multiphoton excitation at (h) 800 nm, and (i) 1200 nm at the delay time of 300 ps. The curves are the best fit to Equation 1, which was used to derive the multiphoton absorption cross-sections.

FIG. 20 refers to a set of images showing the multiphoton excited bioimaging of C. elegans that ingested aqueous HPNCs. (a) is a graph showing the quantitative analysis of worm size following 24 hours exposure to HPNCs treated with InBr₃ and TMOS; (b) is a graph showing the number of worm thrashes per 30 seconds, two hours post-exposure to the HPNCs treated with InBr₃ and TMOS. Each worm population were treated with either vehicle control, HPNCs or sodium azide (worm-paralyzing agent); (c) is a graph showing the total number of eggs laid in each 12-well NGM dish, counted five days after seeding and HPNC exposure. There were 30 worms per sample. T-test: p>0.05. Error bars: standard error (SE). HPNC concentration: 0.5 mg/mL; (d-i) is a set of images showing the bioimaging of C. elegans that ingested HPNCs treated with InBr3 and TMOS under (d) bright field (BF), (e) 400 nm femtosecond laser (repetition frequency: 250 kHz) confocal scan, (f) 400 nm femtosecond laser confocal plus BF scan, (g) BF confocal scan, (h) 1035 nm femtosecond laser (repetition frequency: 50 MHz) scan, and (i) 1035 nm femtosecond laser plus BF scan. Scale bar, 20 μm: (j-n) is a set of images showing the bioimaging of the C. elegans that ingested HPNC samples stored for 96 hours at 4° C., tested under (j) BF, (k) BF confocal scan, (I) 1035 nm scan+HDR mode, (m) 1035 nm plus BF scan plus HDR mode, and (n) 1035 nm 3D scan plus depth coded alpha blending mode. Scale bars, 20 μm.

FIG. 21 refers to a set of images showing the general fluorescence bioimaging of C. elegans that ingested HPNCs treated with InBr₃-0.5 and TMOS. (a-c) are the micro-images of C. elegans that ingested HPNCs under (a) bright field, (b) 488 nm LED excitation, and (c) the merged mode. The micro-images were taken by the Carl Zeiss inverted fluorescence microscope; (d-k) are images showing the bioimaging of C. elegans that ingested HPNCs sample stored for 96 hours at 4° C. at a depth of (d) 0, (e) −5, (f) −7.5, (g) −10, (h) −12.5, (i) −15, (j) −17.5 and (k) −20 μm, upon bright field and 1035 nm femtosecond laser scan. Scale bars, 100 μm.

### DETAILED DISCLOSURE OF OPTIONAL EMBODIMENTS

There is provided a nanocrystal having a core-shell structure, wherein the core of the core-shell structure is at least partially encapsulated by the shell of the core-shell structure, wherein:
the core of the core-shell structure comprises a core perovskite structure having a formula ABX₃, wherein:
   A is selected from the group consisting of at least one ion of one or more group 1 elements of the Periodic Table of Elements, an organic cation having a structure of R¹—(NHₓ)_{y}⁺ wherein R¹ is CH or alkyl, x is 2 or 3 and y is 1 or 2, as valency allows, and any mixture thereof:
   B is at least one ion of one or more group 14 elements of the Periodic Table of Elements; and
   X is a halide ion or any mixture thereof, and
the shell of the core-shell structure comprises a shell perovskite structure and a compound comprising silicon and oxygen, wherein the shell perovskite structure is different from the core perovskite structure and comprises a low-dimensional perovskite structure that is doped with a metal halide comprising a monovalent, divalent or trivalent metal ion.

A may be selected from the group consisting of CH₃NH₃⁺, CH(NH₂)₂⁺, Cs⁺, Rb⁺ and any mixture thereof, B may be selected from the group consisting of Pb²⁺, Sn²⁺, Ge²⁺ and any mixture thereof, and X may be selected from the group consisting of I⁻, Br⁻, Cl⁻, F⁻ and any mixture thereof.

The monovalent, divalent or trivalent metal ion may be an ion of a metal selected from the group consisting of Group IA, Group IIA, Group IIIA or Group IVA of the Periodic Table of Elements, transition metal, lanthanoid series, actinoid series and any mixture thereof.

The monovalent, divalent or trivalent metal ion may be selected from the group consisting of Na⁺, K⁺, Rb⁺, Ca²⁺, Sc³⁺, Cu⁺, Ga³⁺, Cd²⁺, Sn²⁺, Mn²⁺, Y³⁺, Zn²⁺, In³⁺, Ni²⁺, Co²⁺, Al³⁺, Mg²⁺, Fe²⁺, Fe³⁺, Pb²⁺, Bi³⁺, La³⁺, Ce³⁺, Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, Lu³⁺ and any mixture thereof.

For ion doping or substitution in the shell perovskite structure, the monovalent, divalent or trivalent metal ion may improve the functionality of the nanocrystal. Doping or substitution at the A-site (group IA elements of the Periodic Table of Elements) and X-site (halide ions) of the structure ABX₃ may be mainly responsible for adjusting the band gap or emission band, while doping or substitution at the B-site of the structure ABX₃ (except with group IA elements of the Periodic Table of Elements and non-metal elements) may provide additional function to the nanocrystal.

The ion of the metal selected from Group IA of the Periodic Table of Elements may be doped in the A-site of the shell perovskite structure.

The ion of the metal selected from Group IIA, IIIA or IVA of the Periodic Table of Elements, transition metal, lanthanoid series or actinoid series may be doped at the B-site of the shell perovskite structure.

For example, doping with Yb³⁺ may alter f-f transitions and therefore may be useful in quantum cutting applications as it may yield about 200% PLQY in the near-infrared emission, while doping with ions of rare-earth elements may be useful in sensing. Similarly, doping with Mn²⁺ and ions of the iron triad (Fe²⁺, Fe³⁺, Ni²⁺ and Co²) may alter d-d transition emission and magnetic properties and therefore may be useful in spintronics application.

The doping with the metal halide may independently occur on the surface of the core of the core-shell structure, the surface of the core perovskite structure, in the shell of the core-shell structure, in the shell perovskite structure, in the low-dimensional perovskite structure, or any mixture thereof. The shell of the core-shell structure may comprise the shell perovskite structure and the compound comprising silicon and oxygen. The doping may generally occur in the perovskite structure.

The low-dimensional perovskite shell may further comprise a halide ion X′⁻ selected from the group consisting of I⁻, Br⁻, Cl⁻, F⁻ or any mixture thereof.

The halide ion in the low-dimensional perovskite shell may advantageously improve spectral tunability of the nanocrystal. The nanocrystal, depending on the halide used to dope the low-dimensional perovskite structure, may emit across the full colour spectrum, namely between about 350 nm to about 750 nm. By using chlorinated, brominated, or iodinated metal salts, nanocrystals with blue, green or (deep) red emission, respectively may be obtained.

The ratio of the metal ion and halide ion doped in the nanocrystal may not directly correlate to the stoichiometric ratio of the elements comprising the metal halide. The doping amount of the metal ion and halide ion may independently be affected by many factors, including concentration. temperature, volume of doping element and its suitability in the nanocrystal, including valence. As an example, if the nanocrystal is doped with YCl₃, doping with one Y³⁺ ion may not necessarily entail doping with 3 Cl⁻ ions.

The low-dimensional perovskite structure in the shell perovskite structure may be selected from the group consisting of a zero-dimensional perovskite structure, one-dimensional perovskite structure, two-dimensional perovskite structure and any mixture thereof.

The core perovskite structure may comprise three-dimensional γ-CsPbX₃.

The core perovskite structure may comprise substantially of γ-CsPbX₃. The core perovskite structure may comprise greater than 95 wt %, greater than 97 wt % or greater than 99 wt % of γ-CsPbX₃. The core perovskite structure may contain small amounts (less than 5 wt %) of impurities such as Cs₄PbBr₆ which may have been introduced by batch error or room temperature synthesis without an insulating atmosphere. The amount of impurities in the core perovskite structure may be in the range of about 0 wt % to about 5 wt %, about 0 wt % to about 1 wt %, about 0 wt % to about 3 wt %, about 1 wt % to about 3 wt %, about 1 wt % to about 5 wt %, or about 3 wt % to about 5 wt %.

The low-dimensional perovskite structure may comprise a structure selected from the group consisting of zero-dimensional ABX₃ magic sized clusters, zero-dimensional A₄BX₆, one-dimensional δ-ABX₃, two-dimensional ABX₃ nanoplatelets, two-dimensional AB₃X₅ and any mixture thereof.

The one-dimensional δ-ABX₃ may be in the form of nanorods.

When the core perovskite structure comprises three-dimensional γ-CsPbX₃, the low-dimensional perovskite structure may comprise a structure selected from the group consisting of zero-dimensional CsPbX₃ magic sized clusters, zero-dimensional Cs₄PbX₆, one-dimensional δ-CsPbX₃, two-dimensional CsPbX₃ nanoplatelets, two-dimensional CsPb₂X₅ and any mixture thereof.

The presence of the low-dimensional perovskite structures as defined above may be a distinct fingerprint of the nanocrystal as defined above. The low-dimensional perovskite structure may comprise multiple forms of low-dimensional perovskite structures. Further, the low-dimensional perovskite structures in the shell perovskite structure may be different before and after dispersing the nanocrystal in water, or after doping with different metal halides.

When the nanocrystal is dispersed in a substrate such as water, the low-dimensional perovskite structure may become dispersed throughout the substrate.

When the nanocrystal is dispersed in a substrate such as water, the low-dimensional perovskite structure may comprise zero-dimensional CsPbX₃ magic sized clusters (MSCs). The zero-dimensional CsPbX₃ MSCs may have a size in the range of about 2 nm to about 4 nm, about 2 nm to about 3 nm or about 3 nm to about 4 nm.

Magic-sized clusters (MSCs) may be a specific molecular-scale arrangement of atoms that may exhibit enhanced stability. They may grow in discrete jumps, creating a series of crystallites, without the appearance of intermediate sizes.

The compound comprising silicon and oxygen may be selected from the group consisting of silica, silicate, an oligomeric silicon-oxygen compound, siloxane and any mixture thereof.

The nanocrystal may have a particle size in the range of about 40 nm to about 80 nm, about 40 nm to about 50 nm, about 40 nm to about 60 nm, about 40 nm to about 70 nm, about 50 nm to about 60 nm, about 50 nm to about 70 nm, about 50 nm to about 80 nm, about 60 nm to about 70 nm, about 60 nm to about 80 nm or about 70 nm to about 80 nm.

The core of the core-shell structure of the nanocrystal may have a size in the range of about 10 nm to about 30 nm, about 10 nm to about 15 nm, about 10 nm to about 20 nm, about 10 nm to about 25 nm, about 15 nm to about 20 nm, about 15 nm to about 25 nm, about 15 nm to about 30 nm, about 20 nm to about 25 nm, about 20 nm to about 30 nm, or about 25 nm to about 30 nm.

The core of the core-shell structure may all have the same size as defined above. Having the size as defined above may reduce energy dissipation due to size dispersion in the nanocrystals. This may enable the perovskite nanocrystals to achieve optical gain (or stimulated emission) more easily. That is, the nanocrystal may achieve low threshold simulated emission.

There is also provided a process of preparing the nanocrystal as defined above, comprising a step of simultaneously mixing in a mixing solvent, a core perovskite structure having a formula ABX₃, a metal halide comprising a monovalent, divalent or trivalent metal ion and a precursor compound comprising silicon and oxygen, wherein:
A is selected from the group consisting of at least one ion of one or more group 1 elements of the Periodic Table of Elements, an organic cation having a structure of R¹—(NHₓ)_{y}⁺ wherein R¹ is CH or alkyl, x is 2 or 3 and y is 1 or 2, as valency allows, and any mixture thereof:
B is at least one ion of one or more group 14 elements of the Periodic Table of Elements; and
X is a halide ion.

Prior to the mixing step, the process may further comprise the step of dissolving the metal halide in a polar solvent comprising an alcohol, a fatty acid, a fatty amine, and an amine having a structure N(R²)₃, wherein R² may be independently hydrogen or alkyl.

The polar solvent may comprise about 50% to about 80%, about 50% to about 60%, about 50% to about 70%, about 60% to about 70%, about 60% to about 80%, or about 70% to about 80% by volume of the alcohol,

The polar solvent may comprise about 15% to about 25%, about 15% to about 17%, about 15% to about 20%, about 15% to about 22%, about 17% to about 20%, about 17% to about 22%, about 17% to about 25%, about 20% to about 22%, about 20% to about 22% or about 22% to 25% by volume of the fatty acid

The polar solvent may comprise about 5% to about 15%, about 5% to about 7%, about 5% to about 10%, about 5% to about 12%, about 7% to about 10%, about 7% to about 12%, about 7% to about 15%, about 10% to about 12%, about 10% to about 15% or about 12% to about 15% by volume of the fatty amine

The polar solvent may comprise and about 1% to about 3%, about 1% to about 1.5%, about 1% to about 2%, about 1% to about 2.5%, about 1.5% to about 2%, about 1.5% to about 2.5%, about 1.5% to about 3%, about 2% to about 2.5%, about 2% to about 3% or about 2.5% to about 3% by volume of the amine having the structure N(R²)₃.

The total volume of the polar solvent may add to 100%.

The alcohol may be selected from the group consisting of methanol, ethanol, isopropanol and any mixture thereof.

The fatty acid may be a C₂ to C₂₀ fatty acid. The fatty acid may be a saturated or unsaturated fatty acid. The fatty acid may be a monounsaturated fatty acid. The fatty acid may be oleic acid, dodecanoic acid, octanoic acid, hexanoic acid, acetic acid, or any mixture thereof.

The fatty amine may be a C₅ to C₂₀ fatty amine. The fatty amine may be a saturated or unsaturated fatty amine. The fatty amine may be a monounsaturated fatty amine. The fatty amine may be oleylamine, dodecylamine, octylamine, hexylamine or any mixture thereof.

The amine having a structure N(R²)₃, wherein R² may be independently hydrogen or alkyl, may be selected from the group consisting of ammonia, methylamine, dimethylamine, trimethylamine, ethylamine, diethylamine, triethylamine, and any mixture thereof.

The polar solvent may comprise oleic acid, oleylamine, ammonia and an alcohol selected from the group consisting of methanol, ethanol, isopropanol and any mixture thereof.

The metal halide may be selected from the group consisting of NaX′, KX′, RbX′, CaX′₂, ScX′₃, CuX′, GaX′₃, CdX′₂, SnX′₂, MnX′₂, YX′₃, ZnX′₂, InX′, NiX′₂, CoX′₂, AlX′₃, MgX′₂, FeX′₂, FeX′₃, PbX′₂, BiX′₃, LaX′₃, CeX′₃, PrX′₃, NdX′₃, PmX′₃, SmX′₃, EuX′₃, GdX′₃, TbX′₃, DyX′₃, HoX′₃, ErX′₃, TmX′₃, YbX′₃, LuX′₃, and any mixture thereof, wherein X′ is independently selected from Cl, Br, I or F.

The metal halide may be present at a concentration in the range of about 0.1 mM to about 400 mM, about 0.1 mM to about 0.4 mM, about 0.1 mM to about 1 mM, about 0.1 mM to about 4 mM, about 0.1 mM to about 10 mM, about 0.1 mM to about 40 mM, about 0.1 mM to about 100 mM, about 0.4 mM to about 1 mM, about 0.4 mM to about 4 mM, about 0.4 mM to about 10 mM, about 0.4 mM to about 40 mM, about 0.4 mM to about 100 mM, about 0.4 mM to about 400 mM, about 1 mM to about 4 mM, about 1 mM to about 10 mM, about 1 mM to about 40 mM, about 1 mM to about 100 mM, about 1 mM to about 400 mM, about 4 mM to about 10 mM, about 4 mM to about 40 mM, about 4 mM to about 100 mM, about 4 mM to about 400 mM, about 10 mM to about 40 mM, about 10 mM to about 100 mM, about 10 mM to about 400 mM, about 40 mM to about 100 mM, about 40 mM to about 400 mM, or about 100 mM to about 400 mM.

In the mixing solvent, the metal halide may be present at a concentration in the range of about 0.3 mM to about 10 mM, about 0.3 mM to about 0.5 mM, about 0.3 mM to about 1 mM, about 0.3 mM to about 5 mM, about 0.5 mM to about 1 mM, about 0.5 mM to about 5 mM, about 0.5 mM to about 10 mM, about 1 mM to about 5 mM, about 1 mM to about 10 mM, or about 5 mM to about 10 mM.

In the polar solvent, the metal halide may be present at a concentration in the range of about 20 mM to 400 mM, about 20 mM to about 40 mM, about 20 mM to about 100 mM, about 20 mM to about 200 mM, about 40 mM to about 100 mM, about 40 mM to about 200 mM, about 40 mM to about 400 mM, about 100 mM to about 200 mM, about 100 mM to about 400 mM or about 200 mM to about 400 mM.

The precursor compound comprising silicon and oxygen may be selected from the group consisting of tetramethylorthosilicate (TMOS), tetraethylorthosilicate (TEOS), tetraisopropyl orthosilicate (TIPOS), (3-aminopropyl)triethoxysilane (APTES) and any mixture thereof.

The precursor compound comprising silicon and oxygen may be present at a concentration in the range of about 2 mM to about 10 mM, about 2 mM to about 4 mM, about 2 mM to about 6 mM, about 2 mM to about 8 mM, about 4 mM to about 6 mM, about 4 mM to about 8 mM, about 4 mM to about 10 mM, about 6 mM to about 8 mM, about 6 mM to about 10 mM or about 8 mM to about 10 mM.

Prior to the mixing step, the process may further comprise the step of size-sieving the core perovskite structure to a size in the range of about 10 nm to about 30 nm, about 10 nm to about 15 nm, about 10 nm to about 20 nm, about 10 nm to about 25 nm, about 15 nm to about 20 nm, about 15 nm to about 25 nm, about 15 nm to about 30 nm, about 20 nm to about 25 nm, about 20 nm to about 30 nm, or about 25 nm to about 30 nm.

The step of size-sieving may be performed by centrifugation.

The size-sieving of the core perovskite structure to the size as defined above before silica encapsulation may reduce energy dissipation due to size dispersion in the nanocrystals. This may enable the perovskite nanocrystals to achieve optical gain (or stimulated emission) more easily. That is, the nanocrystal may achieve low threshold simulated emission. In contrast, when encapsulation with the silicon-oxygen compound is simultaneously accompanied by the nucleation and growth of the perovskite nanocrystals, it may be difficult to perform size sieving by means of centrifugation and thereby develop perovskite nanocrystals as low threshold optical gain medium.

The core perovskite structure may be present at a concentration in the range of about 25 nM to about 70 nM, about 25 nM to about 30 nM, about 25 nM to about 30 nM, about 25 nM to about 40 nM, about 25 nM to about 50 nM, about 25 nM to about 60 nM, about 30 nM to about 40 nM, about 30 nM to about 50 nM, about 30 nM to about 60 nM, about 30 nM to about 60 nM, about 40 nM to about 50 nM, about 40 nM to about 60 nM, about 40 nM to about 70 nM, about 50 nM to about 60 nM, about 50 nM to about 70 nm, or about 60 nM to about 70 nM.

The mixing solvent may comprise a solvent selected from the group consisting of an alcohol, a fatty acid, a fatty amine, n-hexane, toluene, dichloromethane, an amine having a structure N(R²)₃ wherein R² is independently hydrogen or alkyl, and any mixture thereof.

The mixing step may be performed at a temperature in the range of about 25° C. to about 35° C., about 25° C. to about 27° C., about 25° C. to about 30 ° C., about 25° C. to about 32° C., about 27° C. to about 30° C., about 27° C. to about 32° C., about 27° C. to about 35° C., about 30° C. to about 32° C., about 30° C. to about 35° C. or about 32° C. to about 35° C.

The mixing step may be performed for a duration in the range of about 1 hour to about 36 hours, about 1 hour to about 6 hours, about 1 hour to about 12 hours, about 1 hour to about 24 hours, about 6 hours to about 12 hours, about 6 hours to about 12 hours, about 6 hours to about 24 hours, about 6 hours to about 36 hours, about 12 hours to about 24 hours, about 12 hours to about 36 hours, or about 24 hours to about 36 hours.

There is also provided a substrate comprising the nanocrystal as defined above, wherein the substrate may be selected from the group consisting of an aqueous solution, film, microcrystal, or bulk single crystal.

The film may comprise a polymer selected from the group consisting of polymethyl methacrylate, polyethylene, polyethylene terephthalate (PET), polypropylene (PP), polydimethylsiloxane (PDMS) and any mixture thereof.

There is also provided the use of the nanocrystal as defined above or the substrate as defined above in LEDs, multi-photon imaging, full-colour displays, lasers, bioimaging, optoelectronics, spintronic devices, solar cells, or as radiation detectors.

The bioimaging may be multi-colour and/or multi-functional imaging. In bioimaging, the nanocrystal as defined above may be used as a high fluorescent biomedical label.

The laser may be a low-threshold multi-colour laser.

### Examples

Non-limiting examples of the invention will be further described in greater detail by reference to specific Examples, which should not be construed as in any way limiting the scope of the invention.

### Materials

Lead bromide (22 98%), cesium bromide (99.999%), oleylamine (70%), oleic acid (90%), methyl acetate (anhydrous, 99.5%), ethyl acetate (anhydrous, 99.8%), hexane (anhydrous, 95%), toluene (anhydrous, 99.8%), N,N-dimethylformamide (anhydrous, 99.8%), dimethyl sulfoxide (anhydrous, ≥99.9%), ammonium hydroxide solution (28% NH₃ in H₂O, ≥99.99%), ethyl alcohol (anhydrous, >99.5%), isopropyl alcohol (anhydrous, 99.5%), tetraethyl orthosilicate (99.999%), tetraisopropyl orthosilicate and metal halides for post-treatment were purchased from Sigma-Aldrich (St. Louis, Missouri, USA). Tetramethyl orthosilicate (>99.0%) was purchased from Tokyo Chemical Industry Co., Ltd (TCI, Tokyo, Japan). Reagents were used as such unless until mentioned further purification.

### Methods

### Structural Characterization

Fourier-transform infrared (FTIR) spectroscopy spectra were taken in attenuated total reflection mode (ATR) using a commercial FTIR spectrometer (Invennio-R, Bruker, Billerica, Massachusetts, USA) equipped with diamond ATR accessory. The setup was constantly purged with dry N₂ gas.

X-ray photoelectron spectroscopy (XPS) measurements were conducted using an XPS Shimadzu Kratos Axis Supra (Shimadzu, Kyoto, Japan), with XPS peak information analysed via the National Institute of Standards and Technology (NIST) X-ray Photoelectron Spectroscopy Database. Powder X-ray Diffraction (PXRD) patterns were measured using a PANalytical X'Pert Pro X-ray diffraction system (PANalytical Inc., Malvern, UK) with monochromatic Cu Kα irradiation (λ=1.5418 Å).

The High-Resolution Transmission Electron Microscopy (HR-TEM) and High-Angle Annular Dark-Field Scanning Transmission Electron Microscopy (HAADF-STEM) images were recorded in a ARM300 TEM (JEOL, Tokyo, Japan) equipped with a probe and an image corrector. The HAADF-STEM images were recorded with a 90 mrad semi-angle inner collection angle. The Scanning Transmission Electron Microscopy Energy Dispersive X-ray (STEM-EDX) signal was recorded using a JEOL-EDX detector installed on the ARM300 TEM (JEOL, Tokyo, Japan). The HAADF-STEM and STEM-EDX data were collected at cryogenic temperature to mitigate the contamination using a dedicated micro-electromechanical system (MEMS) based Transmission Electron Microscopy (TEM) holder with improved spatial stability. The spatial drift of the STEM-EDX stack was compensated by realigning the EDX stack using HyperSpy Python plugin.

### Optical Measurements

The optical measurements for multiphoton excited femtosecond transient absorption (TA) experiments were performed by using a Phasetech spectrometer (PhaseTech Spectroscopy. Inc., Madison. Wisconsin, USA). The Near Infrared (IR) pump pulse was generated from an optical parametric amplifier (NDFG, Light Conversion) that was pumped by a 1 kHz regenerative amplifier (Astrella, Coherent Inc. (Santa Clara, California. USA), 35 fs, 1 kHz, 800 nm), with 3.5 mJ input pulse energy. The system was seeded by a mode-locked Ti-sapphire oscillator (Vitesse. Coherent Inc., 80 MHZ). The white light continuum probe beam was generated by focusing a small portion (about 10 μJ) of the regenerative amplifier's fundamental 800 nm laser pulses into a 2 mm sapphire crystal (for visible range). The probe beam was collected using a Charge-coupled device (CCD) sensor (Teledyne e2v. Chelmsford, UK). Time-Resolved Photoluminescence (TRPL) measurements were performed using excitation pulses from the same femtosecond laser system described above. The luminescence signal from the sample was dispersed by a DK240) 1/4 meter monochromator with 150 g mm⁻¹ grating, and the temporal evolution of the photoluminescence (PL) was resolved by an Optoscope streak camera system (Optronics, Kehl, Germany), which has an ultimate temporal resolution of about 10 ps when operated at the shortest time window of 330 ps. Steady-state absorption spectra were collected using a UV-3600 UV-VIS-NIR spectrophotometer (Shimadzu. Kyoto. Japan). Photoluminescence Quantum Yield (PLQY) and Commission Internationale de I'Elcairage (CIE, the International Commission on Illumination) chromaticity measurements were performed using a Jobin-Yvon Fluorolog system (Horiba, Kyoto, Japan) equipped with iHR320 monochromator, coupled with a photomultiplier tube and a spectrally calibrated Spectralon-coated integrating sphere (Quanta-Phi, Lauderdale, Florida, USA). Excitation energy was varied by selecting different components of a Xe lamp emission with a monochromator. Dilute solutions of the samples were contained in a quartz 1 cm×1 cm cuvette.

### C. Elegans Maintenance and Assessment of HPNCs Toxicity and In-Vivo PL Imaging

Caenorhabditis elegans (C. elegans) maintenance and assessment of HPNCs toxicity and in-vivo PL imaging are as follows. The C. elegans wild-type strain (purchased from University of Minnesota Caenorhabditis Genetics Center) was used in all toxicity experiments. C. elegans were maintained on Nematode Growth Medium (NGM) agar plates seeded with Escherichia coli OP50 as food source at 20° C. Worms were synchronized by bleaching in all experiments. A stock solution of 4 mg/ml HPNCs diluted in ultrapure water was prepared and diluted accordingly (from 0.01 mg/mL to 0.5 mg/mL) for various experiments. Toxic effects were assessed by three factors which were body length. locomotion, and reproduction rate. There was negligible toxic effects on the C. elegans animal model at concentrations of up to 0.5 mg/mL HPNCs in toxicity endpoint assays. Bright field images were acquired with Lumar. V12 stereomicroscope (Carl Zeiss AG, Oberkochen, Germany). The worm length was measured using ImageJ software. Locomotion behaviours were assessed by counting worm body thrashes in liquid M9 media. Fifty age-synchronized adult worms were seeded on NGM agar-coated 12-well plates. Reproductive rates were obtained by counting the number of eggs per well 5 days after seeding and exposure to HPNCs. Each experiment noted above was independently repeated three times. In-vivo PL imaging was taken by the Carl Zeiss inverted fluorescence microscope, and were performed after C. elegans ingested HPNCs for 10 minutes.

### Single-Photon to Multi-Photon Excited Bioimaging

Single-photon to multi-photon excited bioimaging was conducted using a 1035 nm femtosecond pulse as the excitation (50 MHz, pulse width about 260 fs) generated from a fully automated ultrafast laser system (Monaco 1035-80-60, Coherent, Inc., Santa Clara, California, USA). In addition, the 400 nm (3.1 eV, 250 kHz, <160 fs) excitation pulse was generated in optical parametric amplifier (Coherent Inc., OPA 9400/9800 series) by simply frequency doubling the 800 nm femtosecond pulses with BBO nonlinear crystal. The 800 nm femtosecond pulses were generated from regenerative amplifier (Coherent Inc., RegA 9000, 250 kHz, <160 fs), which used a Verdi G for CW pumping and Vitara for seed pulses. The incident 400 nm or 1035 nm laser beam was directly coupled to the Nikon C2si-SH C2 Scanner mounted on the Nikon Ti2-E Inverted Motorized Microscope (Nikon, Tokyo, Japan), then focusing on the sample through an objective lens (CFI TU Plan FLUOR Epi 20×, NA/WD: 0.45/4.5 mm). Photo multiplier tubes (PMTs) were used to collect PL signals from the sample for imaging. In addition, the bright field images were taken by a CMOS camera (Zelux® 1.6 MP CMOS Cameras, Thorlabs, Inc., New Jersey, USA) mounted on the microscope. Various functions for bioimaging such as high dynamic range (HDR) mode, Z-stack scanning, and 3D displaying modes were obtained from Nikon NIS-Elements C Software Suite.

### TA Saturation Method for Determining Single- to Multi-Photon Absorption Cross-Section

In order to determine the multi-photon absorption cross section (MPAC, σₙ, where n is the order of MPE) of these aqueous HPNCs, the transient absorption (TA) saturation method was developed to uniformly solve the measurement of single- to multi-photon excitation. This was a method for directly measuring the multiphoton absorption cross section, which was different from the existing commonly used multiphoton excited PL ratio method that can only perform indirect measurement.

By analysing the bleaching signal |ΔOD|ₜ_{ₗ} at the exciton absorption peak in the TA data of the optically diluted sample at a sufficiently long pump-probe delay time (tₗ), the Poisson distribution could be used to uniformly describe the related dynamic processes generated after single-/multi-photon excitation:

$$\\ \begin{matrix}{\vert Δ OD \vert}_{{t}_{l}} = a ( 1 - {e}^{{}_{} - 〈 N 〉} ) & ( 1 )\end{matrix}$$

$$\\ 〈 N 〉 = {σ}_{n} {F}_{n} = {{σ}_{n} ( \frac{{P}_{peak}}{ℏ ω} )}^{n} τ / n$$

in which, α is a constant that relates to instrumental and sample parameters. [symbol] N [symbol] refers to the average number of photons (for single photon excitation) or equivalent high-energy photons (for multiphoton excitation) absorbed per QD at the given excitation fluence. σₙ (n=1, 2, 3, 4, 5, . . . ) refers to the single-/multi-photon absorption cross-section (in cm²ⁿ sⁿ⁻¹photons¹⁻ⁿ), and Fₙ refers to the photon fluence for single photon excitation or equivalent photon fluence for multiphoton excitation (in cm⁻²photonsⁿs¹⁻ⁿ) at the given excitation wavelength.

Further, Pₚₑₐₖ is the peak power density, ℏω is the photon energy for excitation and τ is the laser pulse width. By plotting Fₙ and |ΔOD|ₜ_{ₗ}, and fitting the data using Equation 1, the value of σₙ can be derived. This method did not require information such as the size and concentration of the NCs and was a means of directly obtaining single-/multi-photon absorption cross-sections.

### Example 1: Synthesis

CsPbBr₃ nanocrystals were first synthesized as the template before performing post-treatment with a metal halide salt solution and tetramethyl orthosilicate (TMOS) simultaneously to achieve full-colour emitting halide perovskite nanocrystals (HPNCs) with both high photoluminescence quantum yield (PLQY), stability and dispersibility in water. In contrast to conventional methods where organic halides with high solubility is used in low-polar solvents for post-treatment of perovskite nanocrystals to tune the emission spectrum, in the present disclosure, a mixed polar solvent system of alcohol/oleic acid (OAc)/oleylamine (OAm)/ammonia was used to prepare a clear solution of metal halide salts with high concentration (0.02 to 0.4 M) for post-treatment.

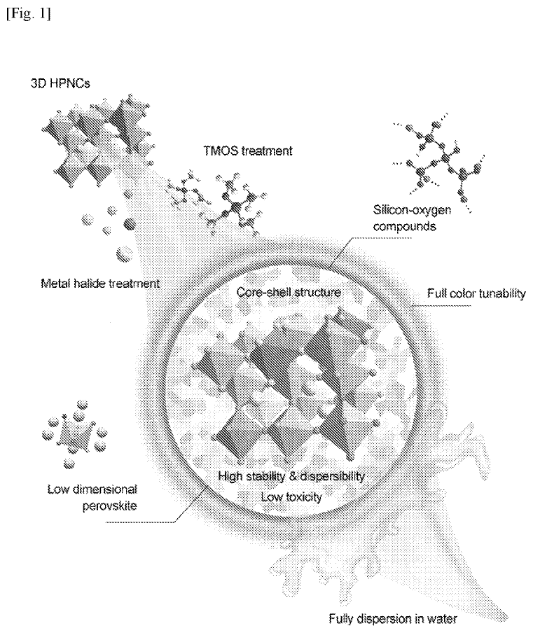

This method contravened conventional wisdom, as non-polar or low polarity solvents are typically used in these post-treatment steps instead of a polar solvent, to minimize any potential damage to the HPNCs. Surprisingly, this method not only overcame the issue of poor solubility of most metal halides such as MnCl₂, YbCl₃, CoX₂, and InX₃, where X=Cl⁻, Br⁻, I⁻, in low-polar solvents, but also allowed effective functional ion doping or substitution of the shell structure. More importantly, the method enabled self-repairing of the thus produced HPNCs, and resulted in the formation of a halogen-rich, low-lead, low-dimensional perovskite shell layer on the surface of the template CsPbBr₃. This perovskite shell layer bound with the silicon-oxygen compound provided by the hydrolysis of TMOS, to synergistically facilitate stronger passivation and better protection from environmental stressors such as water, oxygen, heat or irradiation, as well as to improve the dispersibility of the nanocrystals in water and to reduce the toxicity of the nanocrystals (FIG. 1).

### Preparation of the Template CsPbBr₃ Nanocrystals

CsPbBr₃ nanocrystals (NCs) were synthesized by the ligand-assisted re-precipitation (LARP) method. Briefly, PbBr₂ (73.4 mg, 0.2 mmol), CsBr (42.6 mg, 0.2 mmol), oleic acid (0.5 mL), and oleylamine (0.25 mL) were added to 5 mL dimethylformamide (DMF) or a (9:1) v/v DMF/Dimethyl sulfoxide (DMSO) mixed solution and stirred to be fully dissolved. A portion (1 mL) of the obtained mixture was swiftly injected into 10 mL toluene under vigorous stirring. After stirring, the resultant solution was subjected to a combination of centrifugation, which removed small-sized crystals at high speed (8000 rpm for 10 minutes) and large-sized crystals at low speed (3000 rpm for 15 minutes). The final size-sieved nanocrystals were stored in n-hexane for further use.

### Preparation of the Metal Halide Solution for Post-Treatment

0.1 to 2.0 mmol metal halide salts (MnCl₂, ZnCl₂, NdBr₃, MnI₂, MgI₂, RbBr, CoBr₂, YCl₃, YbCl₃, InBr₃, NiI₂, InI₃, AlI₃ or ZnI₂) were added to 5 mL of a mixed solution (alcohol:oleic acid:oleylamine:ammonia solution=34:10:5:1 by volume) and stirred to be fully dissolved for further use. In the present disclosure, the number following the metal halide in the sample name refers to the molar amount of metal halide used. For example, “InBr₃-0.5” means that 0.5 mmol of InBr₃ was used to prepare the metal halide solution for post-treatment, and the solution was prepared as described above.

Synthesis of the Aqueous Perovskite Nanocrystals with Metal Halide and TMOS Post-Treatment

0.2 mL metal halide solution and 5 μL tetramethyl orthosilicate (TMOS) were added to 10 mL of the approximately 60 nM template CsPbBr, nanocrystals solution and stirred for 2 to 24 hours at room temperature. Then the resultant solution was mixed with ethyl acetate at a volume ratio of 1:1 and centrifuged to obtain a precipitate. Finally, after drying, the precipitate was directly dispersed in water to form an aqueous perovskite nanocrystal solution. It should be noted that in the present disclosure, (½) in the sample name, for example “MnCl₂-1.0 (½)”, means that the concentration of the template CsPbBr₃ solution used in this sample is half of the approximately 60 nM template CsPbBr₃ nanocrystal solution.

### Example 2: Post-Treatment by Metal Halides and TMOS

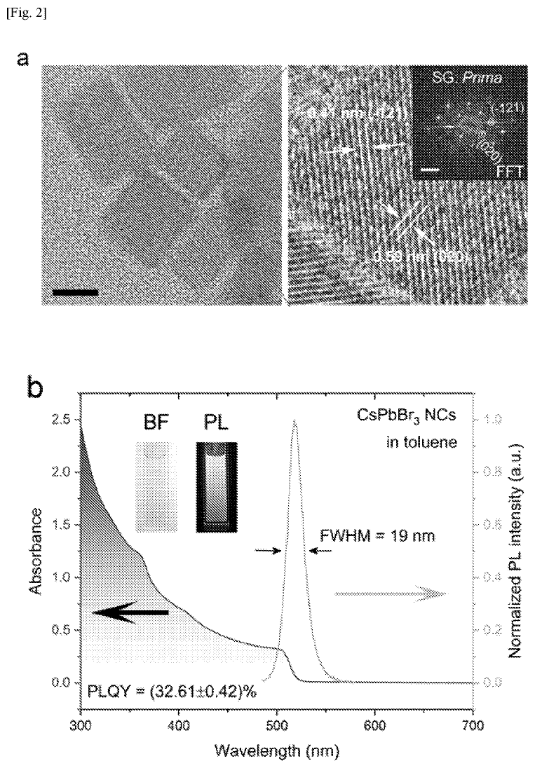

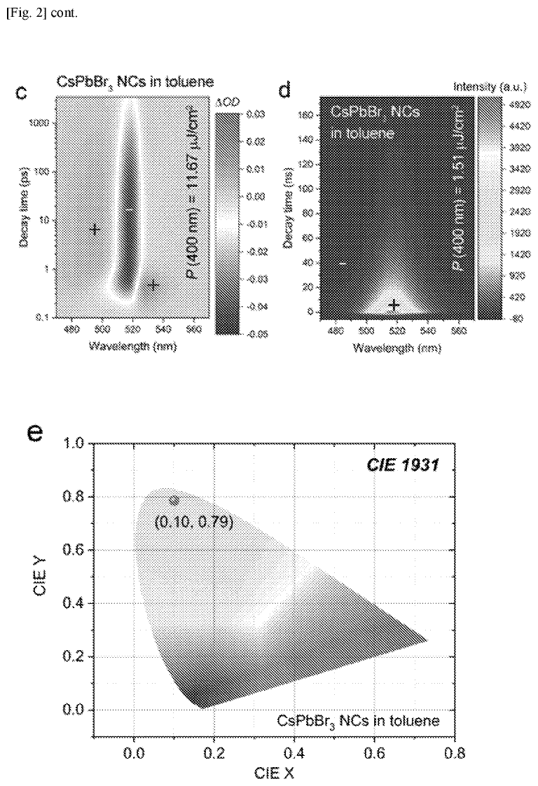

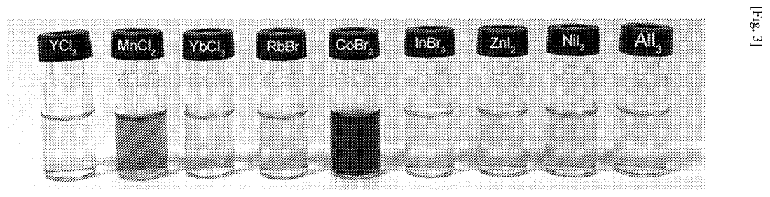

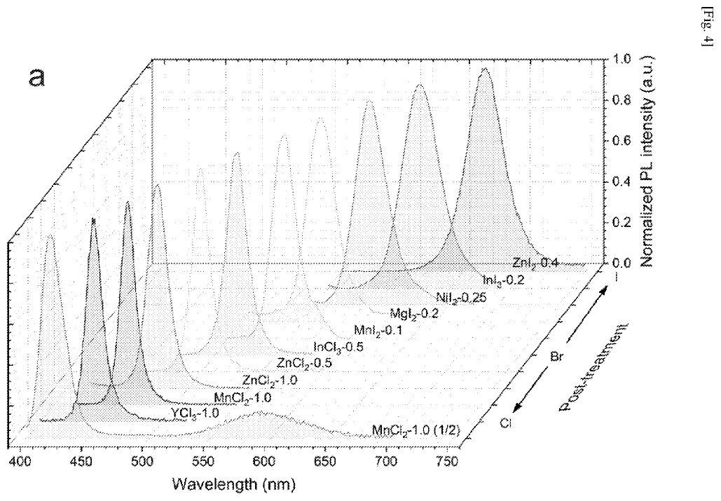

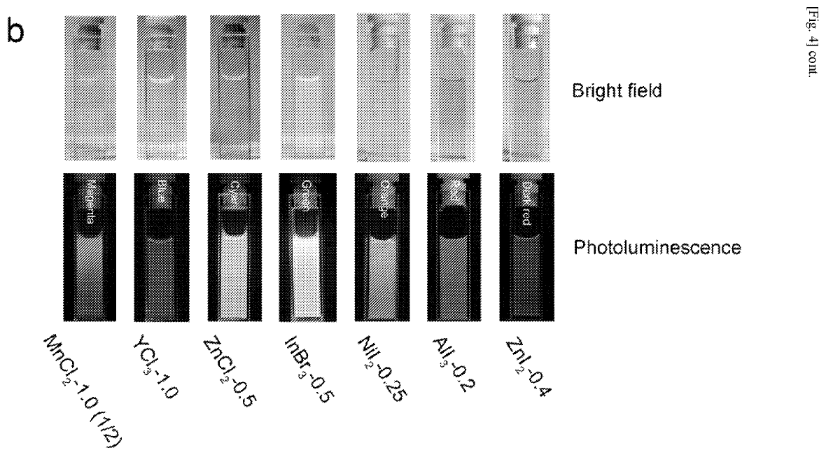

The template CsPbBr₃ nanocrystals were synthesized by the facile, ligand-assisted re-precipitation (LARP) method. Basic characterization including high resolution transmission electron microscopy (HRTEM), absorption and photoluminescence (PL) spectra, as shown in FIG. 2, indicated that the as-synthesized CsPbBr, nanocrystals possessed the Pnma crystal structure, with a rectangular shape and average edge length of 21 (±7) nm (FIG. 2a), and emitted a PL centred at 519 nm, with full width at half maximum (FWHM) of about 19 nm and a PLQY of about 32.6% in toluene (FIG. 2b). FIG. 2c and FIG. 2d show the pseudo-colour transient absorption (TA) spectrum and time-resolved PL (TRPL) spectrum of the as-synthesized CsPbBr₃ nanocrystals, measured in toluene. FIG. 2e indicates the Commission Internationale de l'éclairage (CIE, International Commission on Illumination) chromaticity of the as-synthesized CsPbBr₃ in toluene.

FIG. 3 shows the representative metal halide clear solutions which was used to functionalize the core nanocrystals in multiple ways. The solutions were miscible with the template CsPbBr₃ NC solution, which was typically in low-polar solvents such as toluene and n-hexane, as well as the liquid of silicon-oxygen compounds as such precursors TMOS and (3-aminopropyl)triethoxysilane (APTES). This showed that the developed solvent was a good homogeneous system for ion-doping/substitution and material hybridization of nanocrystals.

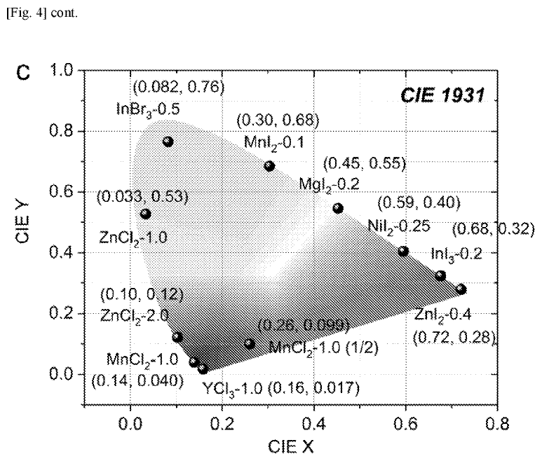

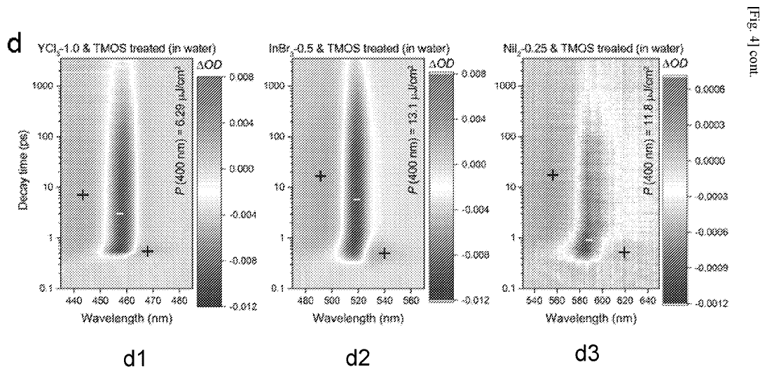

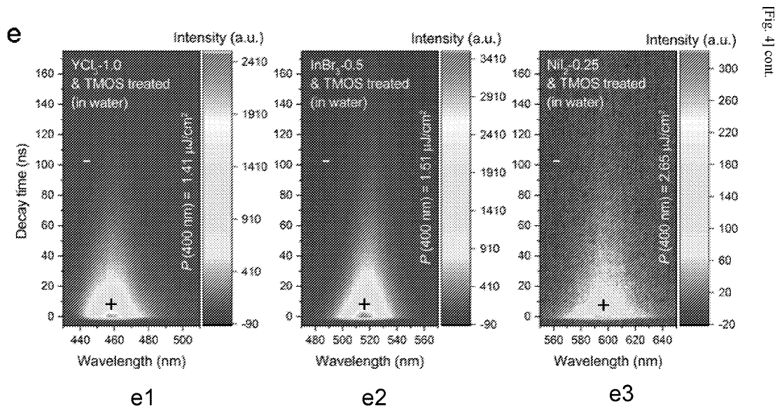

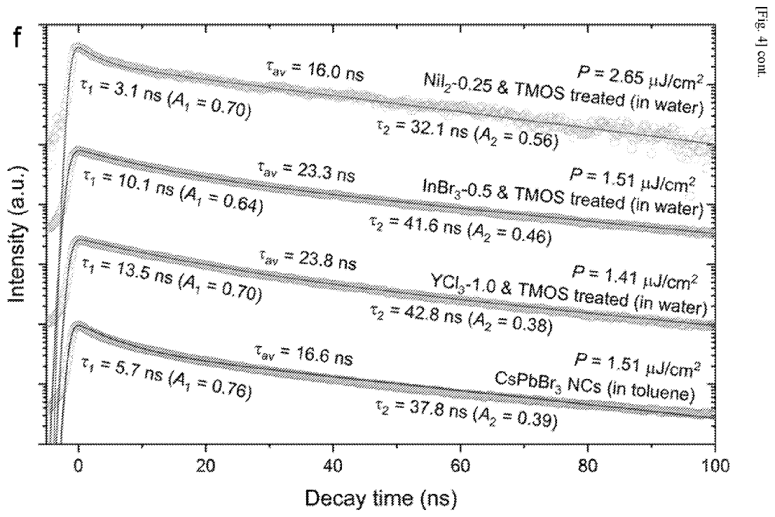

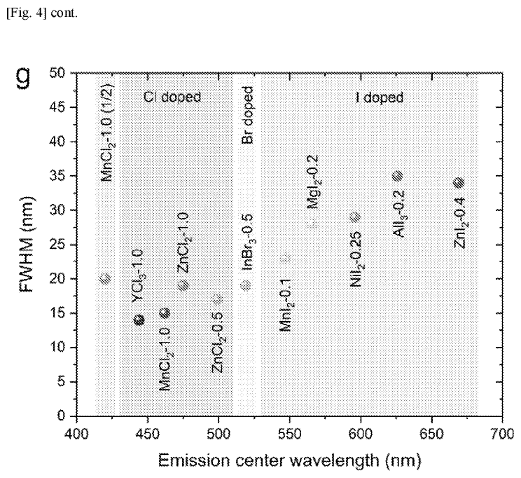

FIG. 4a and FIG. 4c show the PL and CIE chromaticity analysis, respectively, of HPNCs post-treated with representative metal halides and TMOS, measured in water. FIG. 4b shows that the synthesized HPNCs were highly dispersible in water.

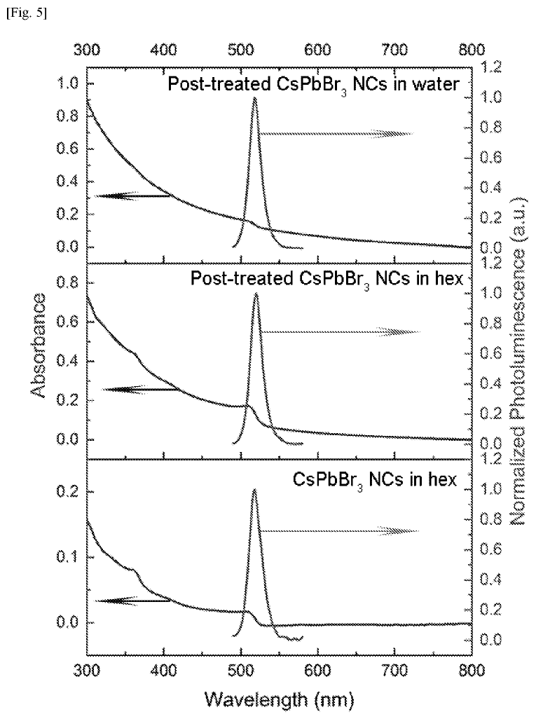

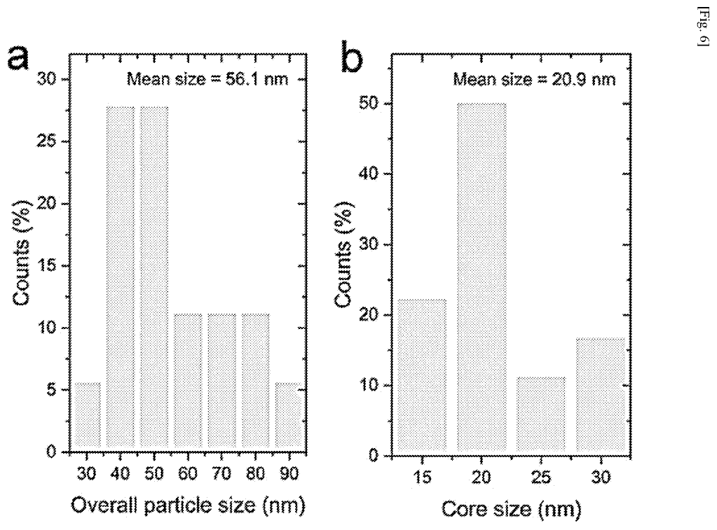

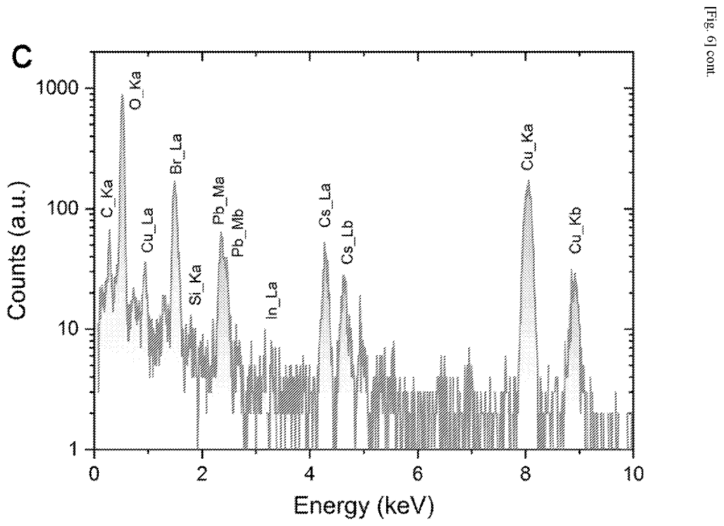

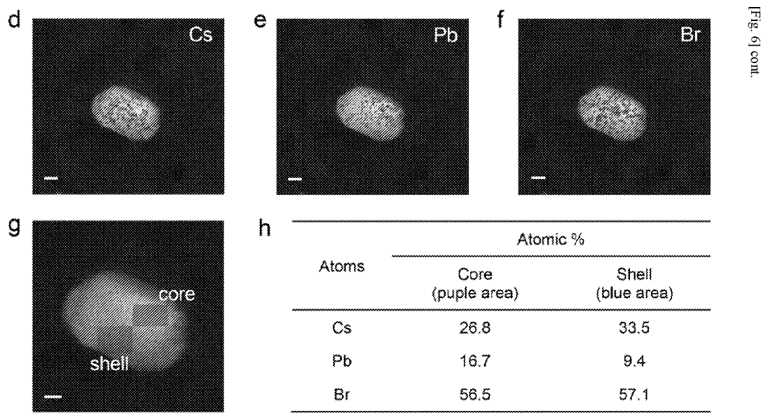

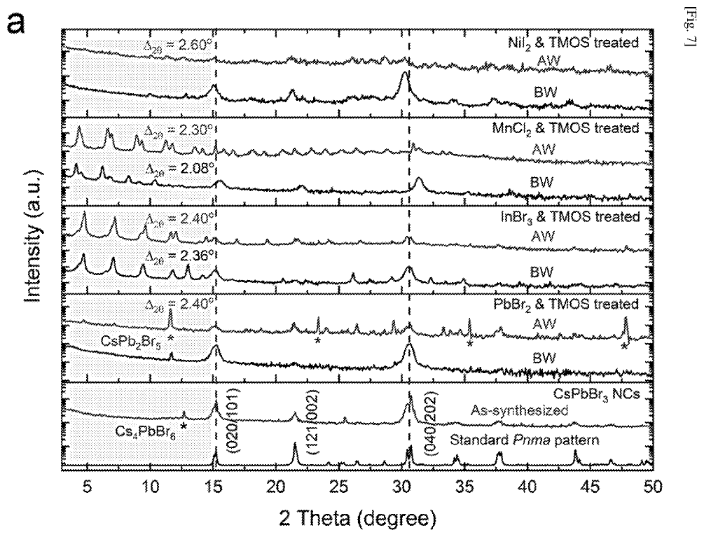

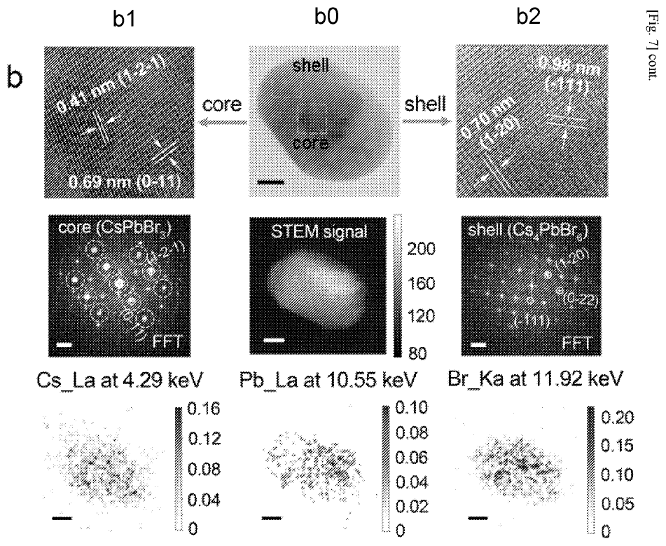

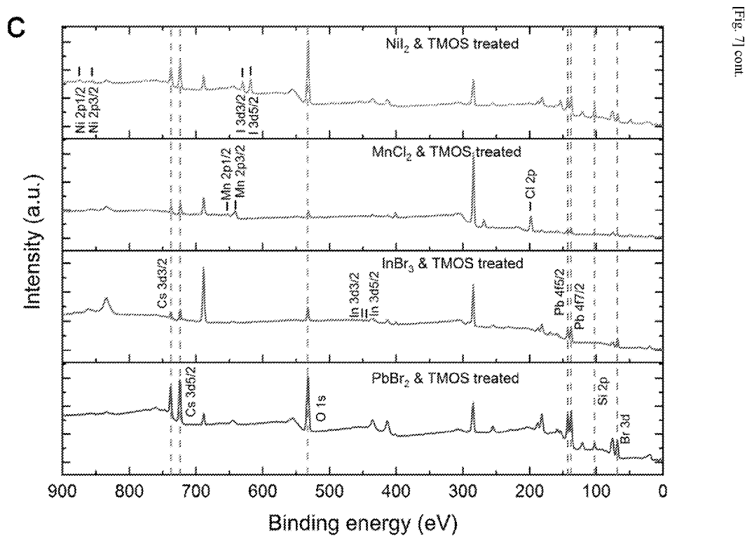

FIG. 5 compares the optical properties of the template CsPbBr₃ nanocrystals and the nanocrystals treated with PbBr₂ and TMOS. The emission peak position and peak width of the nanocrystals before and after the post-treatment step basically remained unchanged.

These perovskite nanocrystals were easily dispersed in water and gave strong emission with high colour purity and wavelength, which was adjustable from about 420 nm to about 670 nm. depending on the metal halide used in the post-treatment. The tunning of the emission properties of these nanocrystals originated from treatment with different metal halide solutions.

Both the A-site ion (alkali metal) and X-site ion (halogen) were mainly responsible for causing band gap adjustment. For example, Cl⁻, Br⁻ or I⁻ treatment causing doping or substitution provided blue, green, or red emission due to doping or substitution at the X-site; while B-site ions (except elements in Group IA of the Periodic Table of Elements and non-metal elements) provided additional emission bands, such as the d-d transitions of transition metals. For example. Mn emission of 500 to 700 nm that is evident in FIG. 4a demonstrated successful Mn²⁺ doping in the resultant HPNCs.

Apart from the different spectral widths of both the transient absorption (TA) bleach and PL emission bands, pseudo-colour TA spectra of pristine CsPbBr₃ NCs in toluene and HPNCs treated with representative metal halides, specifically YCl₃ for blue-emission, InBr₃ for green-emission and NiI₂ for red-emission, and TMOS, as measured in water were rather similar, all showing an initial carrier cooling stage within 2 ps at low pump fluence (P), photoinduced bleach bands for delay times within 4 ns (FIG. 4d and FIG. 2c), and a slight time-delayed redshift in time-resolved PL (TRPL) data, which was recorded with a streak-camera, as shown in FIG. 4e and FIG. 2d.

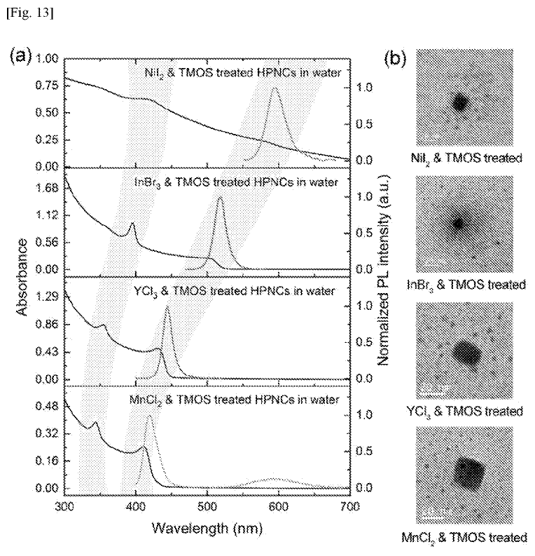

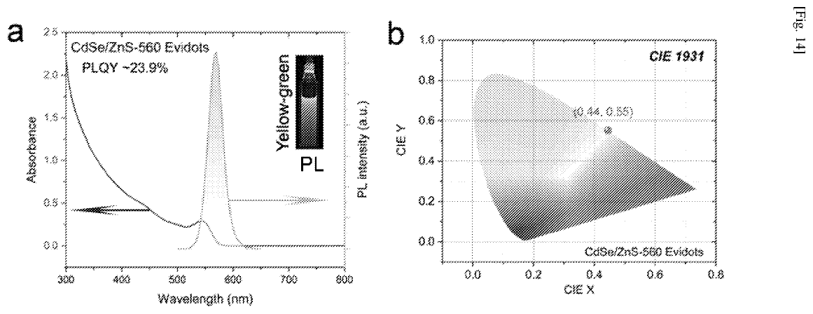

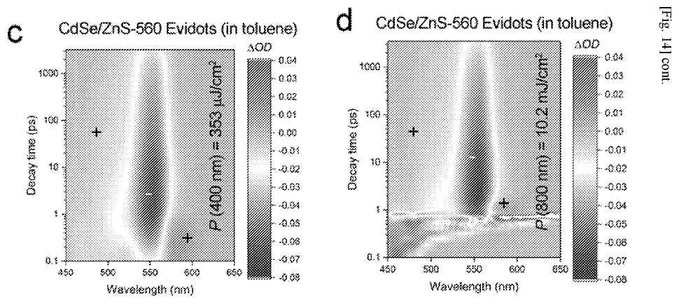

Detailed TRPL decay curves are shown in FIG. 4f, where the post-treated HPNCs in water exhibited a fluorescence lifetime that was comparable to or even significantly longer than that of pristine CsPbBr₃ in toluene (τ_{avg} of about 16.6 ns). All decay curves were fitted with a double-exponential decay. The fast decay was attributed to the direct radiative relaxation of the band-edge excitons while the long-lived decay was ascribed to thermally repopulated band-edge excitons from shallow trap states. The significantly increased PL lifetime as shown in FIG. 4f, the obvious exciton absorption peaks as shown in FIG. 13 and the narrow emission FWHM, as shown in FIG. 4g, all measured in water, indicated the excellent passivation and protection in the HPNCs treated with TMOS and representative blue- and green-emitting YCl₃-1.0 and InBr₃-0.5, respectively. For the red-emitting HPNC series represented by NiI₂-0.25 with TMOS post-treatment, although the performance was slightly worse, including the marginally shortened PL lifetime, weaker exciton absorption peak, and broadened emission FWHM (FIG. 4a, FIG. 4f, FIG. 13 and FIG. 4g), it was still a significant improvement compared to unprotected iodide perovskite nanocrystals, whose fluorescence will typically quench instantly upon moisture contact.

### Example 3: Structural Characterisation

FIG. 7a presents the powder X-ray diffraction (PXRD) patterns of pristine CsPbBr₃ NCs and HPNCs treated with representative metal halides and TMOS. The as-synthesized pristine CsPbBr₃ NC sample mainly displayed the structure of the Pnma space group (SG) in the orthorhombic system, and the small peak of 2θ of approximately equal to 12.7° in the pattern could be attributed to the Cs₄PbBr₆ structure (SG: R-3c). After PbBr₂ and TMOS post-treatment, the main CsPbBr₃ NCs peaks remained, but the Cs₄PbBr₆ peak vanished, accompanied by the appearance of a new peak (about 11.7°) attributed to CsPb₂Br₅ (SG: I4/mcm) formation, which originated from the high levels of Pb²⁺ and Br⁻ in the system.

Further, the introduction of water was thought to contribute to the formation of CsPb₂Br₅ given that the phase of CsPb₂Br₅ was more pronounced while the main peak of the CsPbBr₃ phase was slightly weakened after being dispersed in water.

Using metal chloride (or iodide) for post-treatment, it was found that the diffraction peak of the pristine CsPbBr₃ was still present although it had shifted to a larger angle (or a smaller angle). This was because the lattice constant became smaller (or larger) after being doped with chloride (or iodide) ions.

Interestingly and unexpectedly, the HPNC samples that were post-treated with metal halide and TMOS showed periodic diffraction peaks in the low-angle region (3° to 15°), which was attributed to the formation of low-dimensional perovskites. For the HPNC samples that used lead halide or metal iodide for post-treatment, these small-angle periodic peaks only appeared after being dispersed in water, but they were both weak and difficult to distinguish. This may be because the lead-rich surface was easier to construct high-dimensional structures, while iodide ions were not suitable for stabilizing low-dimensional structures due to their large volume.

In contrast, the non-lead metal bromide and chloride post-treated HPNCs (HPNCs treated with TMOS and InBr₃-0.5 or MnCl₂-1.0 (½)) showed significant periodic peaks either before or after the introduction of water. In particular, the peak position/intensity of the former was basically unchanged, indicating a high structural stability.

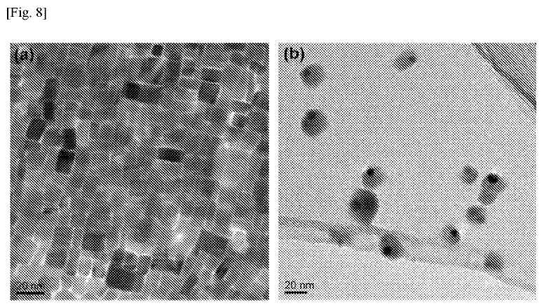

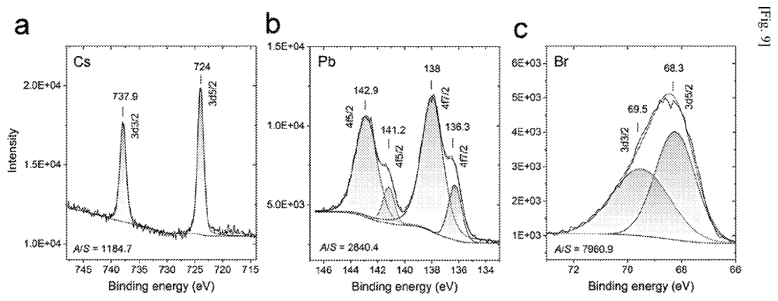

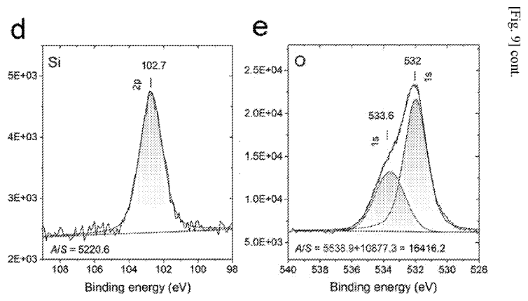

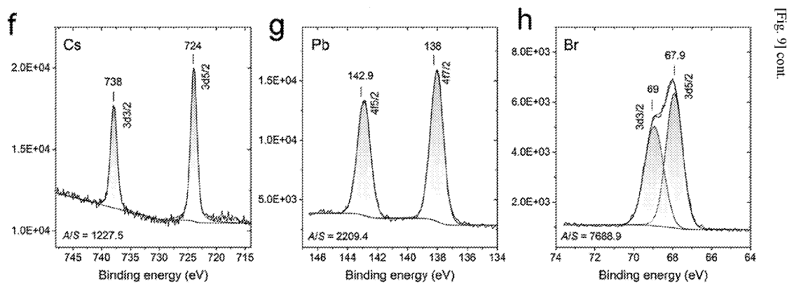

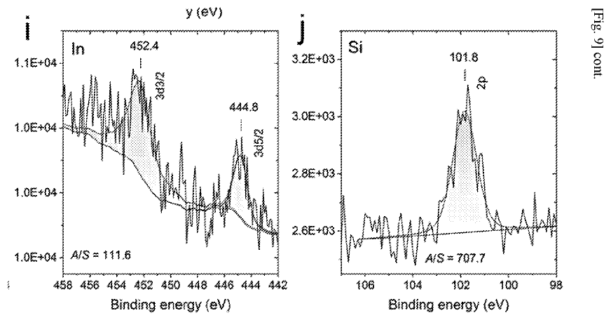

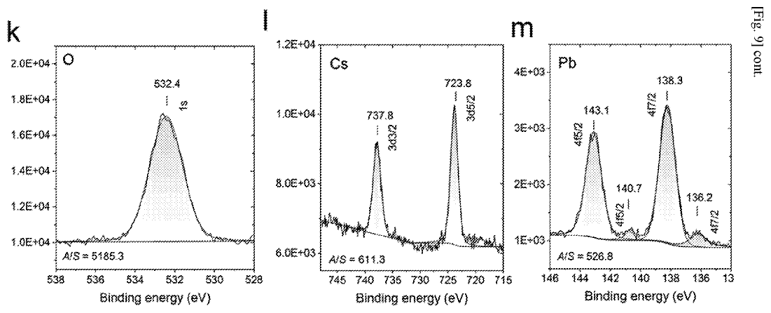

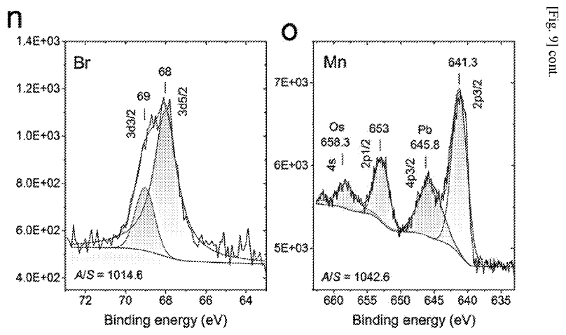

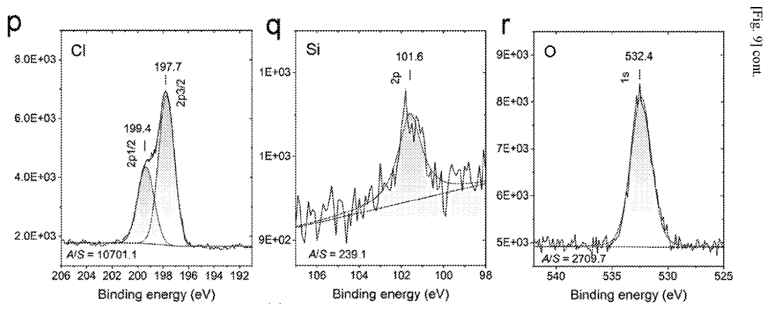

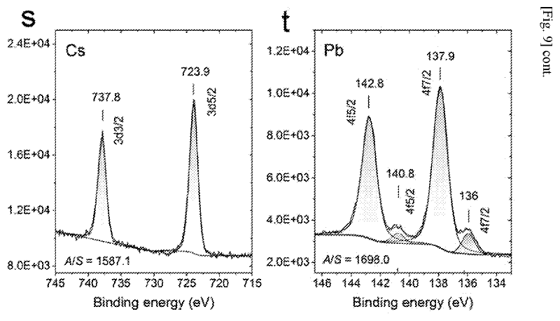

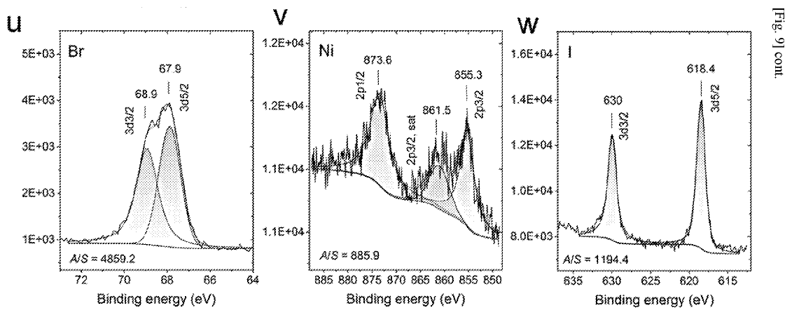

FIG. 8a is a typical transmission electron microscopy (TEM) image of CsPbBr₃ nanocrystals after PbBr₂ post-treatment, in which many nanocrystals exhibited lattice fringes, indicating that good crystalline properties were obtained. FIG. 8b clearly shows the core-shell structure of CsPbBr₃ nanocrystals after post-treatment with PbBr₂ and TMOS.

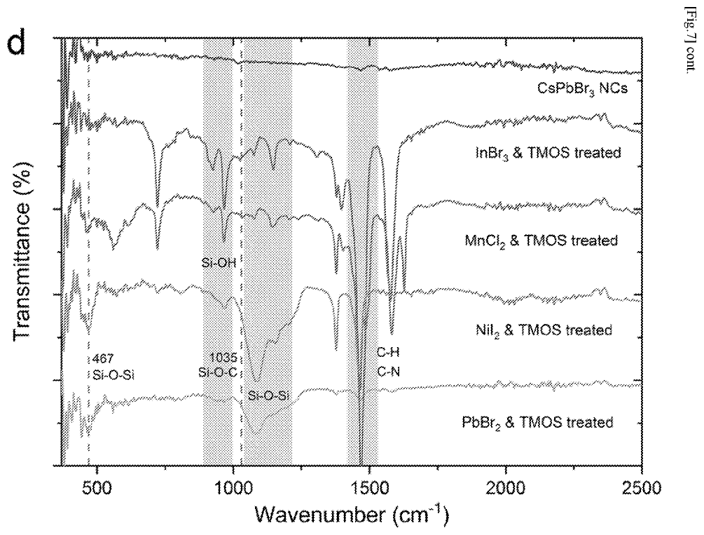

High-resolution electron transmission microscopy (HRTEM) further confirmed that the sample of HPNC treated with InBr₃-0.5 and TMOS, before dispersion in water, had a core-shell structure (FIG. 7b), with an overall average particle size of 60 (±20) nm and an average core size of 21 (±6) nm (FIGS. 6a and 6b). The size of the core CsPbBr₃ was basically the same as the overall dimension of the pristine nanocrystals, indicating that the thick shell did not modify the dimensions of the core of the particle. The shell was imaged in the [211] direction (FIG. 7b2, middle row) and indexed in the trigonal R-3c SG. The lattice spacings of 0.7 nm and 0.98 nm were derived from its associated fast Fourier transform (FFT) pattern and corresponded to the (1-20) and (−111) planes of Cs₄PbBr₆. It did not rule out the doping of In³⁺ as the low doping level would not visibly affect the lattice spacing.

The FFT pattern of the HRTEM image of the core (FIG. 7b1, middle row) consisted of an overlap of the core and the shell lattices, which was consistent with the fact that the core was embedded in the surrounding shell. The strongest reflections were drawn in dotted circles on the FFT and corresponded to the orthorhombic CsPbBr₃ Pnma planes viewed in the [311] direction. The remaining reflections were from the shell as they were identical to the reflections observed in the FFT pattern of the shell (FIG. 7b2, middle row). It was remarkable that the orientation of the shell crystal structure remained unchanged across the whole nanocrystal, indicating that the shell had a single crystal nature.

The nanocrystals were also imaged using high-angle annular-dark-field (HAADF) scanning TEM (STEM) mode coupled with energy dispersive X-ray spectroscopy (EDX) to map the composition of the core-shell structure. It was found that the core had a brighter contrast than the shell in HAADF-STEM mode, indicating a higher density in the core compared to the shell. The EDX mapping indicated that the core had a higher concentration of Pb while Cs and Br were distributed more evenly throughout the particle (FIG. 7b and FIG. 6d-f). In particular, an atomic ratio of Cs:Pb:Br=3.6:1:6.1 in the shell area was obtained by the quantification of the EDX data (FIG. 6g-h), which was consistent with the composition of Cs₄PbBr₆. In addition, In and Si signals were visible in the EDX spectra averaged over the particle, implying the realization of In doping and introduction of silicon-related structures, but just above the noise level (FIG. 6c) due to the low concentration of these two elements and the limited total electron dose used to mitigate electron-beam induced degradation.

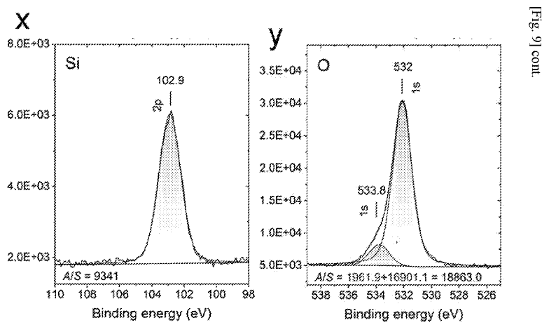

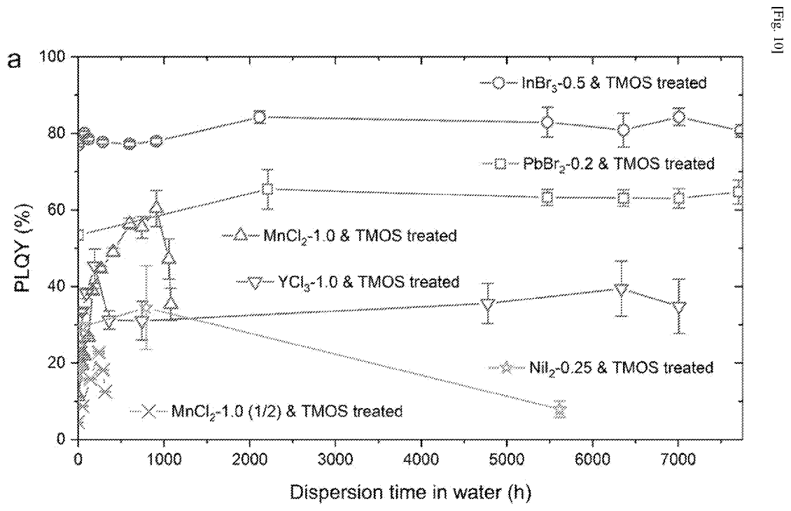

The full scan X-ray photoelectron spectroscopy (XPS) spectra of HPNCs treated with TMOS and representative metal halides (PbBr₂-0.2, InBr₃-0.5, MnCl₂-1.0 (½) and NiI₂-0.25) showed that peaks of Cs 3d, Pb 4f, Br 3d, Si 2p and O 1 s were observed in all cases, while In 3d, Mn 2p and Cl 2p, Ni 2p and I 3d were separately present in the three non-lead metal treated samples (FIG. 7c), which were analysed in detail via high resolution XPS in FIG. 9. By means of XPS peak-differentiation-imitating analysis, it was shown that the high-resolution spectrum of Pb 4f consisted of two major peaks of Pb²⁺ 4f_{5/2} at about 143 eV and Pb²⁺ 4f_{7/2} at about 138 eV and two small shoulder peaks at about 141 eV and about 136.2 eV attributed to the Pb⁰ metallic state. The XPS peak position of Si 2p in the HPNC samples with PbBr₂ or NiI₂ treatment was about 102.8 eV, while it was about 101.7 eV in HPNCs samples treated with InBr₃ or MnCl₂. Similarly, there were also two cases in the O 1 s analysis, which had two XPS peaks of about 533.7 eV and about 532 eV in the former, and only a single peak at 532.4 eV in the latter.

The higher binding energy (Si 2p of 102.8 eV and O 1 s of 533.7 eV) was attributed to the formation of a silica network structure, while the lower binding energy (Si 2p of 101.7 eV and O 1 s of about 532 eV) may be attributed to the Si—O—H or oligomeric Si—O structure and the oxygen from the ligand oleic acid, which were also confirmed by Fourier Transform infrared spectroscopy (FTIR) analysis.

FIG. 7d shows that both the HPNCs treated with TMOS and PbBr₂-0.2 or NiI₂-0.25 were dominated by the vibration of Si—O—Si, verified by the broad band peaking at about 1084 cm⁻¹, while HPNCs treated with TMOS and InBr₃-0.5 or MnCl₂-1.0 (½) mainly possessed the oligomeric Si—O—Si and Si—O—H structures with peaks at about 1147 cm⁻¹ and 900 to 980 cm⁻¹.

All these XPS and FTIR findings echoed the aforementioned PXRD results. Combined with the analysis of the TEM results, they indicated that the structure of these HPNCs before water introduction should be: (a) the non-lead metal chloride/bromide and TMOS post-treatment tended to form a halogen rich low-dimensional perovskite shell, supplemented by oligomeric Si—O—Si and Si—OH structures, which may be the reason for the high dispersibility of the resultant HPNCs in water; and (b) the post-treatment of lead halide/metal iodide and TMOS was not suitable for the formation of low-dimensional perovskite shell, but favoured the formation of a highly polymerized Si—O—Si network structure that protected the perovskite core.

### Example 4: Water, Light and Chromaticity Stability

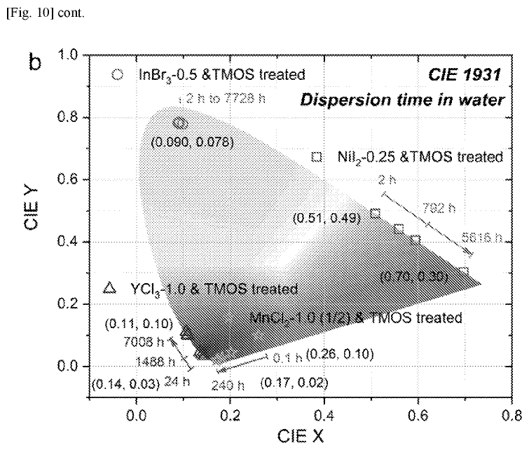

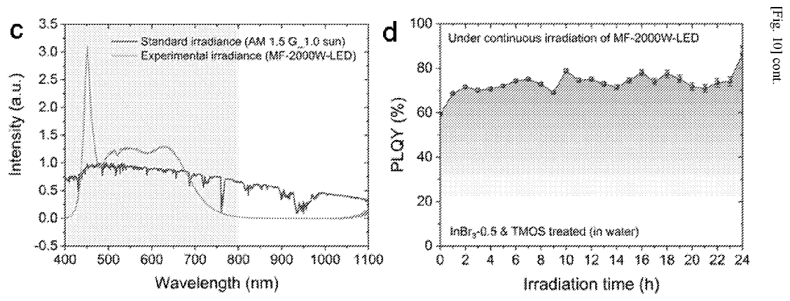

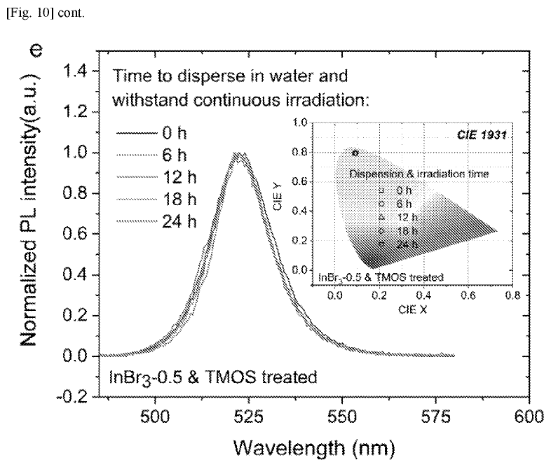

All the post-treated HPNCs showed outstanding stability performance regardless of whether there was formation of a low-dimensional perovskite shell supplemented with oligomeric Si—O—Si and Si—OH structures or a highly polymerized Si—O—Si structure, whereby the former was superior. FIG. 10a shows the dispersion time-dependent PLQY of HPNCs treated with representative metal halides and TMOS, in water. For green emission, the HPNCs treated with PbBr₂-0.2 and TMOS, which mainly formed highly polymerized Si—O—Si structures, maintained a PLQY of >60% after being dispersed in water for >7700 hours. On the other hand, HPNCs treated with InBr₃-0.5 and TMOS which formed a low-dimensional perovskite shell and oligomeric Si—O—Si and Si—OH structures displayed an astonishing PLQY of >80% and high chromatic stability (FIG. 10b) after >7720 hours of being dispersed in water. This was by far the most stable and bright perovskite NC that is known.

Furthermore, when an irradiation source (MF-2000W-LED) calibrated to be equivalent to 1 sun irradiation was used to continuously irradiate an aqueous solution of the HPNCs treated with InBr₃-0.5 and TMOS for 24 hours, the PLQY was maintained at about 80% with the chromaticity remaining invariant, thereby validating high irradiation stability (FIG. 10c-e).

The excellent water/chromaticity/irradiation stability performances indicated that the low-dimensional perovskite shell supplemented with oligomeric Si—O—Si and Si—OH structures provided better passivation and protection than pure, highly polymerized Si—O—Si structure. For the HPNCs post-treated with blue and red emitting metal halide (chloride and iodide, respectively) and TMOS, the stability also dramatically improved. To date, there have not been any reports on the stability of chlorinated or iodinated perovskite NCs in aqueous solution.

The PLQY of HPNCs treated with MnCl₂-1.0 and TMOS which had pure blue emitting peaks at about 462 nm approached 60% after being dispersed in water for more than 900 hours. For HPNCs treated with YCl3-1.0 and TMOS with emission centre wavelength at about 458 nm, the PLQY attained was about 40% (FIG. 10a) and the chromaticity green-shifted slightly (FIG. 10b) after being in water for an amazing >7000 h.

For red-emitting HPNCs, after being dispersed in water for about 750 hours, the HPNC treated with NiI₂-0.25 and TMOS with emission centre wavelength of about 600 nm had a PLQY of about 30% (FIG. 10a), which was accompanied by an increased spread in the error bars and a significant red shift of the CIE chromaticity coordinates (FIG. 10b), implying a decrease in the effective luminescence concentration and the occurrence of iodide migration. Although the red-shift continued with increasing dispersion time in water, these red-emitting HPNCs still retained a PLQY of about 8% after >5600 hours in water, which was a great improvement compared to typical perovskite NCs whose fluorescence would quench almost immediately upon exposure to moisture.

The lower defect tolerance of chloride perovskites and the ion migration in iodide perovskites resulted in lower PLQY than bromide perovskites. The HPNCs treated with MnCl₂-1.0 (½) and TMOS with higher concentration of MnCl₂ showed a maximum PLQY of only about 23% after being dispersed for 250 hours in water (FIG. 10a). This lower PLQY was also attributed to the energy loss to the Mn²⁺ excited state. The slight blue shift of the CIE chromaticity coordinates (FIG. 10b) in this sample also indicated that long-term dispersion in water also affected the stability of Mn²⁺ in the structure.

### Example 5: Water-Induced Phase Transformation of the Shell

It was noted that the PLQY of most HPNCs treated with metal halide and TMOS increased after being dispersed in water (FIG. 10a). This was likely due to the reorganization of some surface structures following post-treatment that led to the formation of halogen rich HPNCs under water induction that reduced related defects, as well as the formation of lead halide hydroxide protective structure.

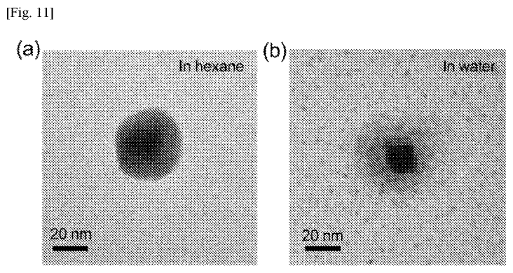

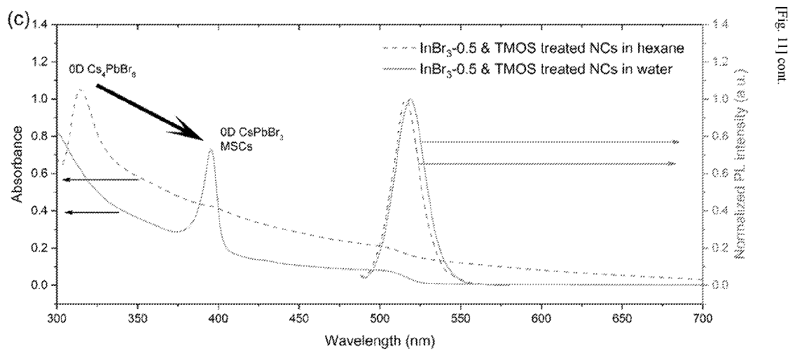

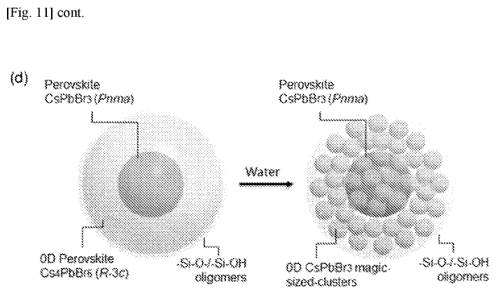

FIGS. 11a and 11b show the typical transmission electron microscopy (TEM) images of HPNCs post-treated with InBr, and TMOS dispersed in hexane and in water, respectively. As discussed above, the as-synthesized HPNCs in hexane had a core-shell structure, where the core was the three-dimensional CsPbBr₃ and the shell was the zero-dimensional Cs₄PbBr₆. After dispersion in water, the structure of the core of the HPNCs was retained while the shell became an assembly of ultra-small nanoclusters. These ultra-small nanoclusters were not only present surrounding the core but also dispersed uniformly in the solution, having an average size of about 3 nm.

FIG. 11c clearly shows the evolution of the absorption-emission spectra of HPNCs from being dispersed in hexane to water. That is, the absorption peak at about 315 nm which is assigned to Cs₄PbBr₆ was completely transformed to the position of about 396 nm, which matched well with the absorption peak of zero-dimensional CsPbBr, magic-sized clusters (MSCs).

FIG. 11d shows a schematic of the possible structural evolution of the core-shell perovskite nanocrystals before and after dispersion in water. After being dispersed in water, the zero-dimensional perovskite Cs₄PbBr₆ in the shell transformed into a large amount of CsPbBr₃ magic sized clusters. In addition, no emission bands of low-dimensional perovskites were observed in the emission spectrum of the nanocrystal, implying that the energy/carrier transfer from the shell to the core was very efficient, which was likely an important reason for the high PLQY of the nanocrystal.

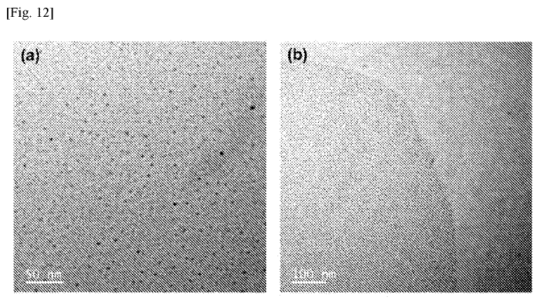

FIG. 12 shows the TEM images of the generated zero-dimensional CsPbBr₃ MSCs in an aqueous system, having a uniformly dispersed form (FIG. 12a) and a two-dimensional assembly form (FIG. 12b). It is generally believed that MSCs are intermediates in the formation of quantum dots and even nanostructures with various dimensions. The existence of MSCs made the nanocrystal system more diverse, as they may make it easier to form low-dimensional perovskite-based structures such as one-dimensional or two-dimensional structures. The low-angle periodic peaks in the PXRD pattern of FIG. 7a likely originated from the low-dimensional layered structure formed by the assembly of these MSCs.

FIG. 13a shows the absorbance and PL spectra of HPNCs post-treated with TMOS and representative metal halides including MnCl₂, YCl₃, InBr₃ and NiI₂. measured in water. The spectra indicated that after different metal halide post-treatment, the shifts of the absorption peaks attributed to MSCs appeared in the same direction as those attributed to three-dimensional perovskites. Similar to three-dimensional perovskites, the blue- or the red-shift of the absorption peaks of the MSCs was dependent more on chemical composition rather than size, given that the MSCs had similar sizes of about 3 nm (FIG. 13b).

### Example 6: Multiphoton Excitation Performance in Water

The stable and bright full-range colour emitting HPNCs enabled extremely challenging MPE measurements to be performed directly in water, which was unprecedented due to the thigh pump fluence needed.

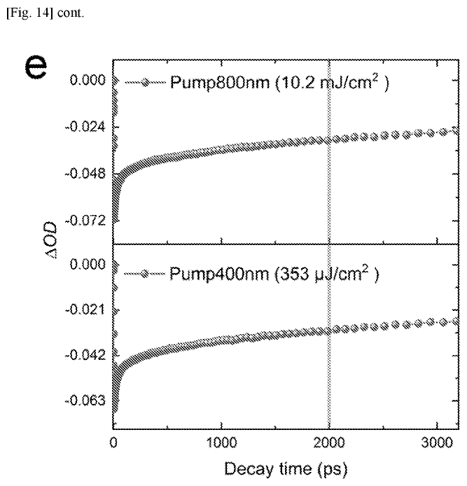

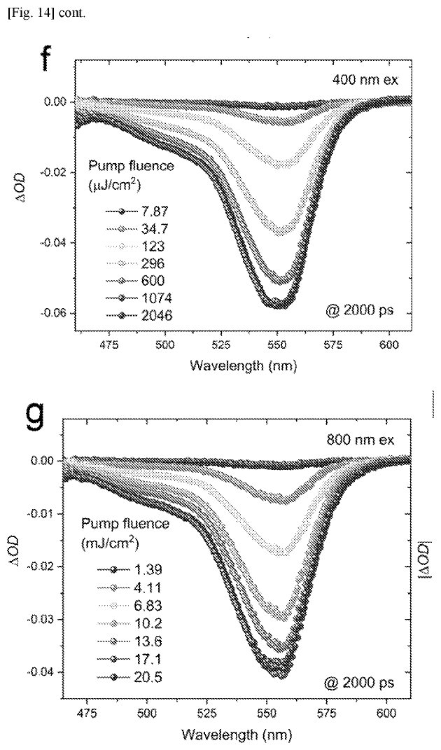

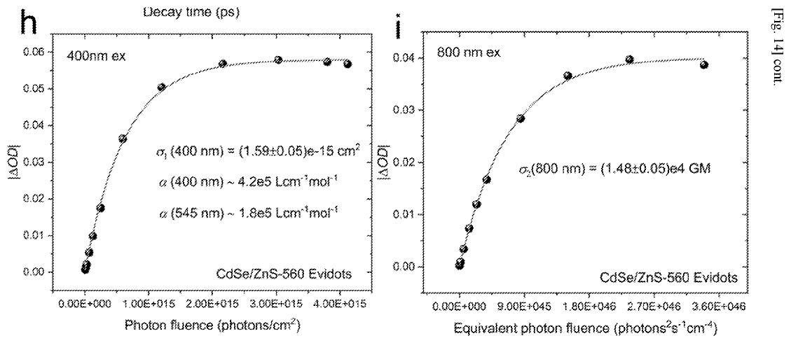

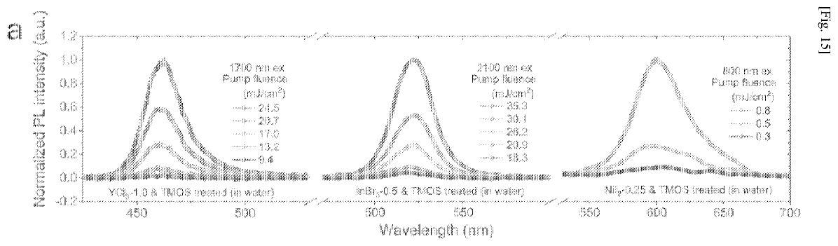

The commercially available quantum dots (QDs) CdSe/ZnS with the trade name of ED-C11-TOL-0560 from Evident Technologies was used as the reference sample to evaluate the reliability of this method. FIG. 14 shows the basic optical characterization of this commercial CdSe/ZnS QDs and the saturation trend of the TA spectral bleaching signal under single-photon (400 nm) to two-photon (800 nm) excitation with increasing pump fluence, which could be highly fitted by Equation 1 to derive σ₁ (400 nm) and σ₂ (800 nm) to be 1.59×10⁻¹⁵ cm² and 1.48×10⁴ GM, respectively.

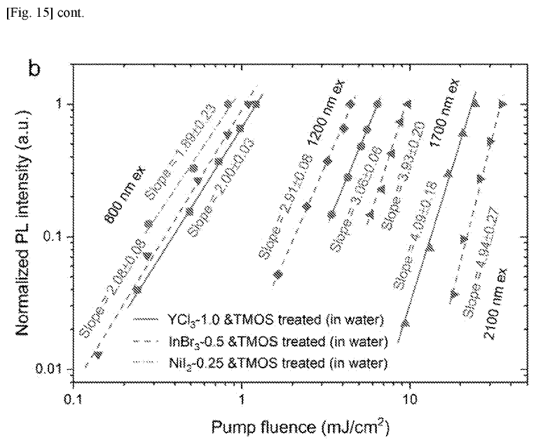

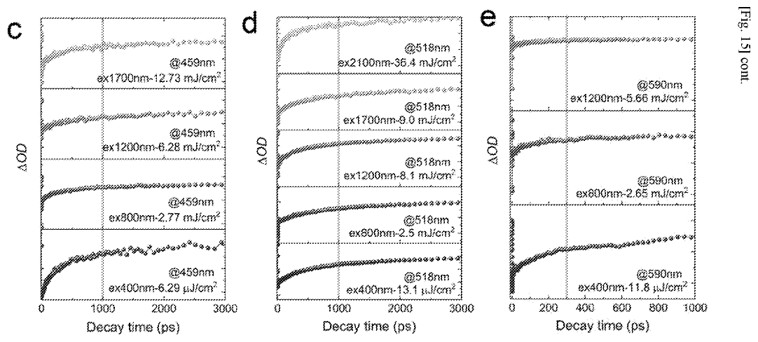

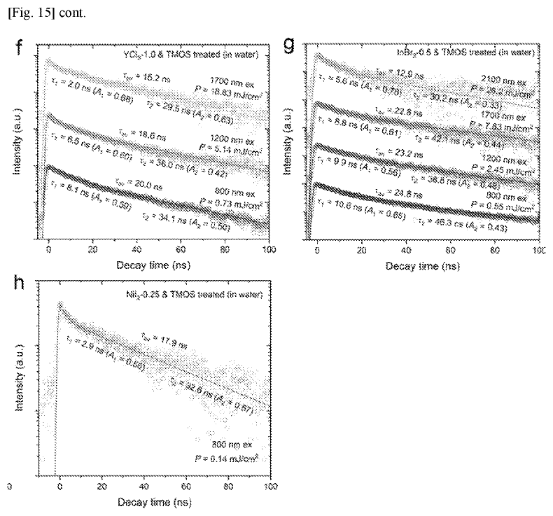

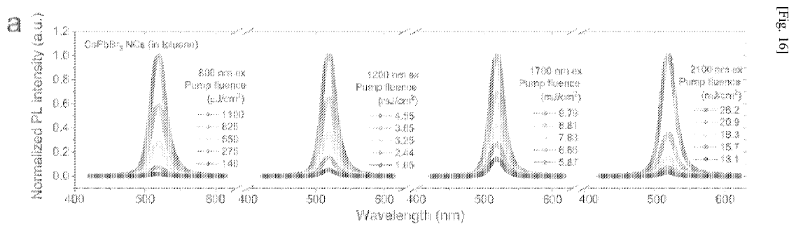

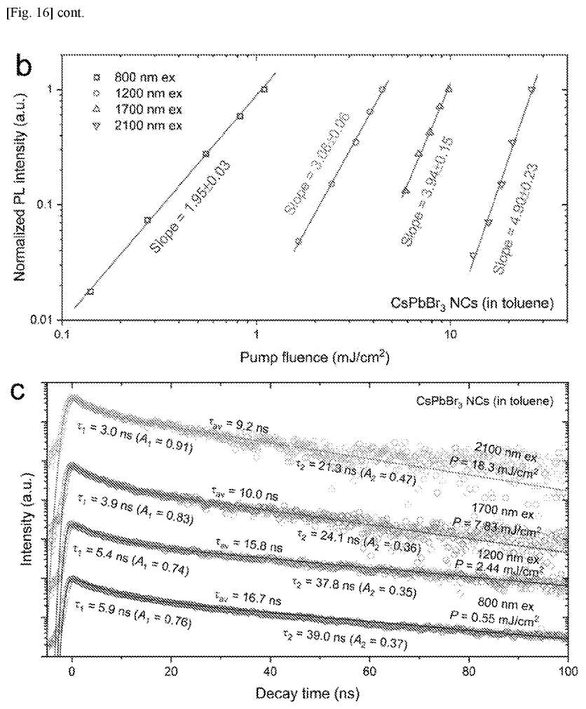

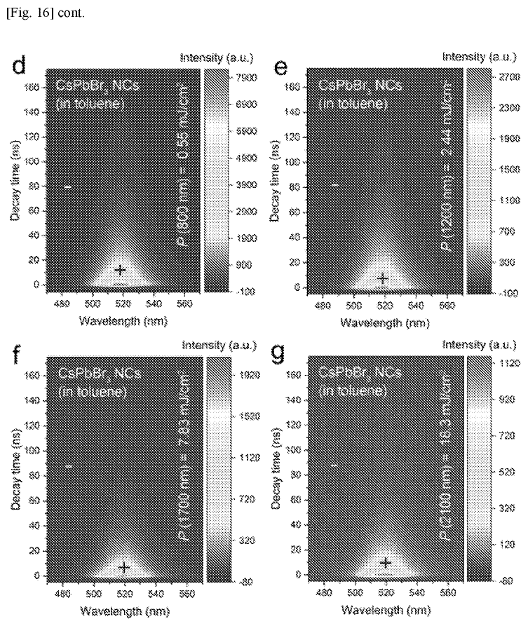

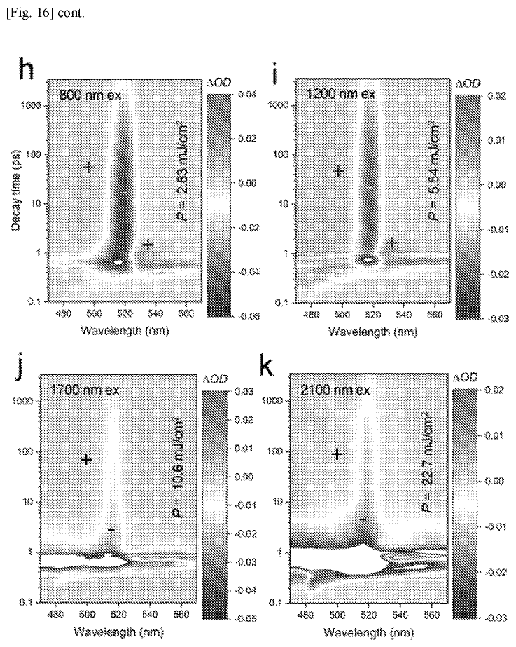

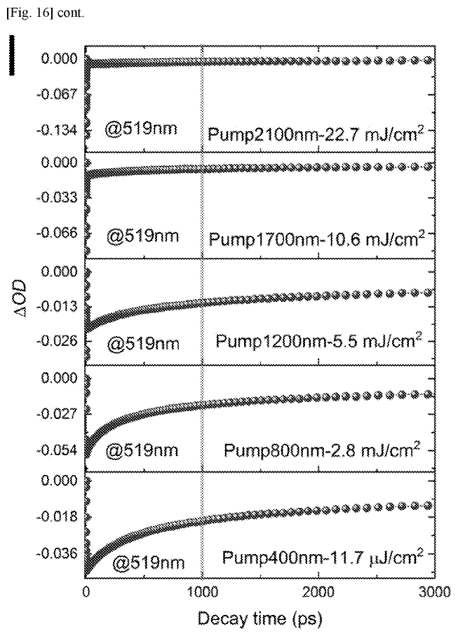

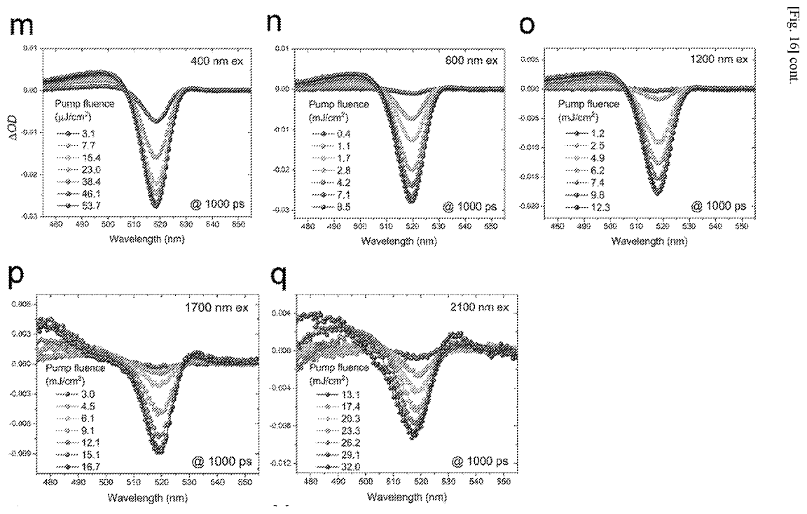

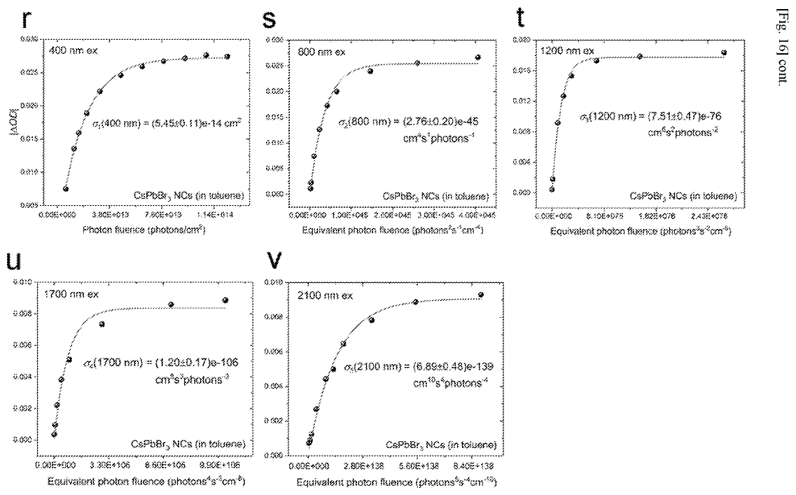

The dynamics of the two-dimensional TA spectroscopy of multi-photon excitation was basically the same as that of single-photon excitation (FIG. 16h-k, 17e-g, 18f-i, and 19b-c), especially since both of them left only TA signals reflecting the single exciton participation under the long pump-probe delay time (FIG. 15c-e, FIG. 16l), which indicated that the properties and dynamics of transient species, such as excitons, produced in the material after single-photon or multi-photon excitation were similar.

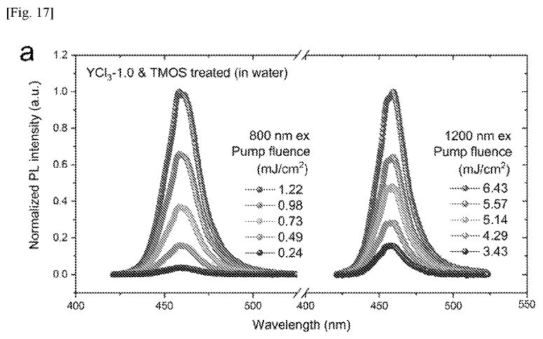

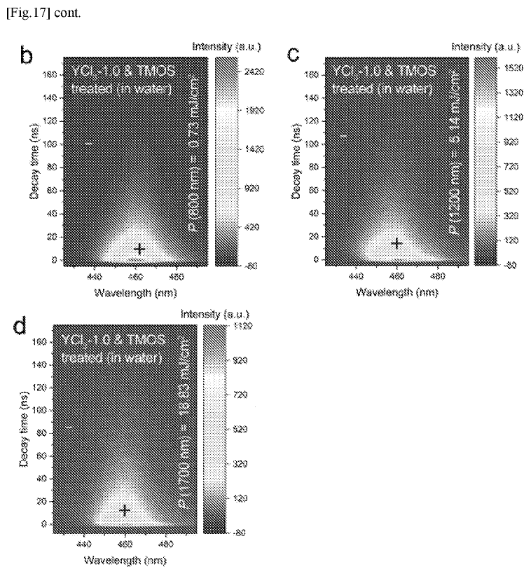

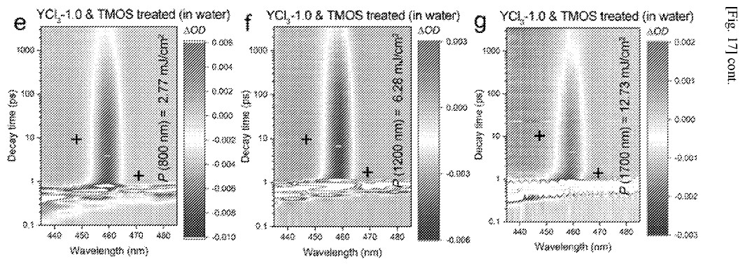

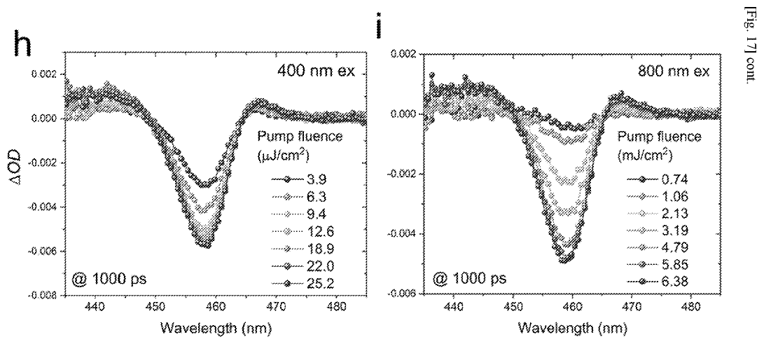

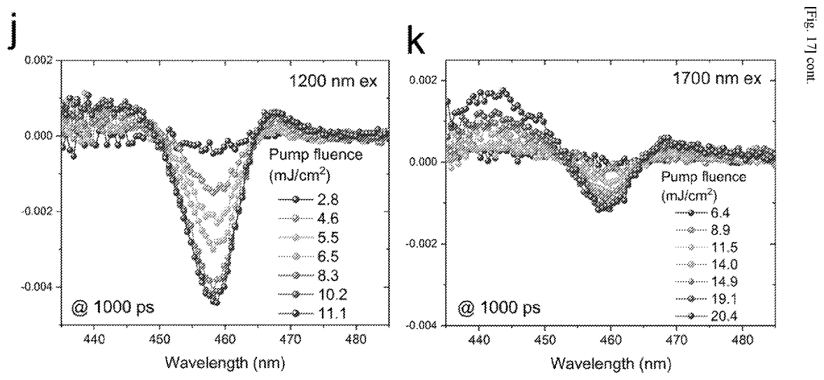

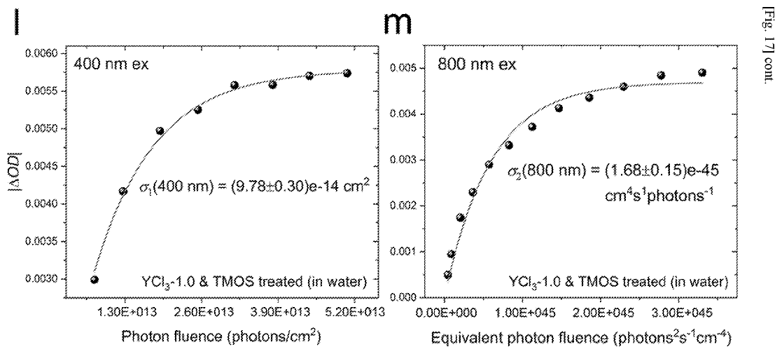

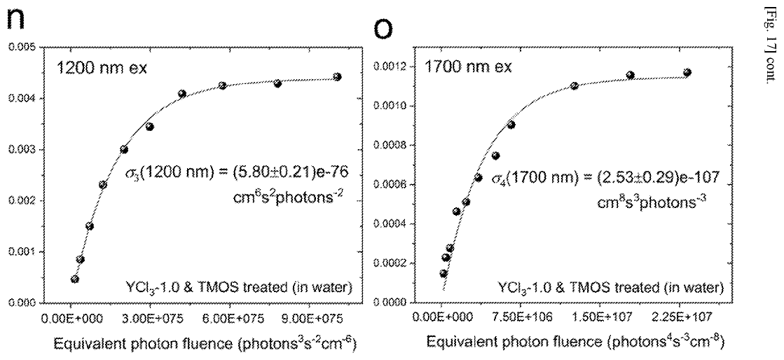

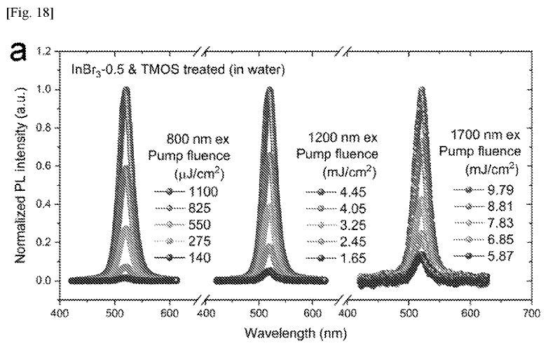

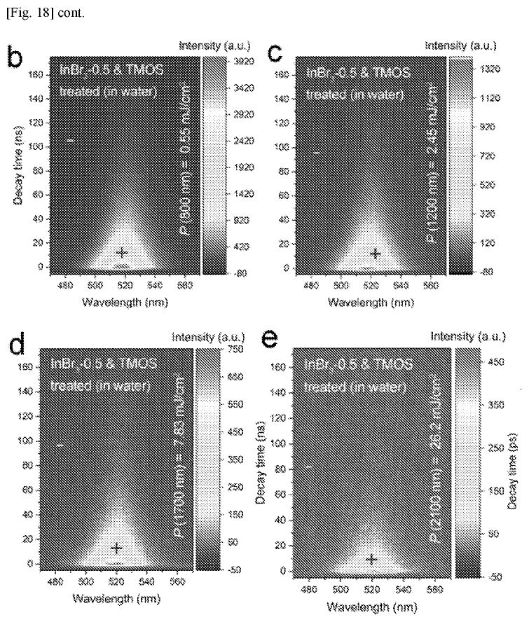

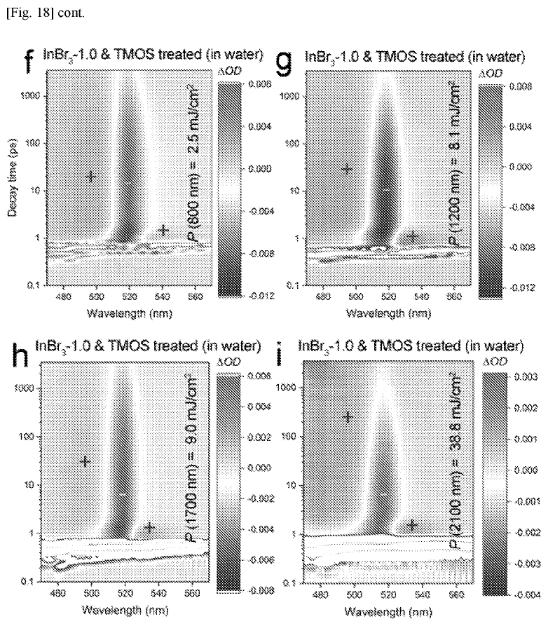

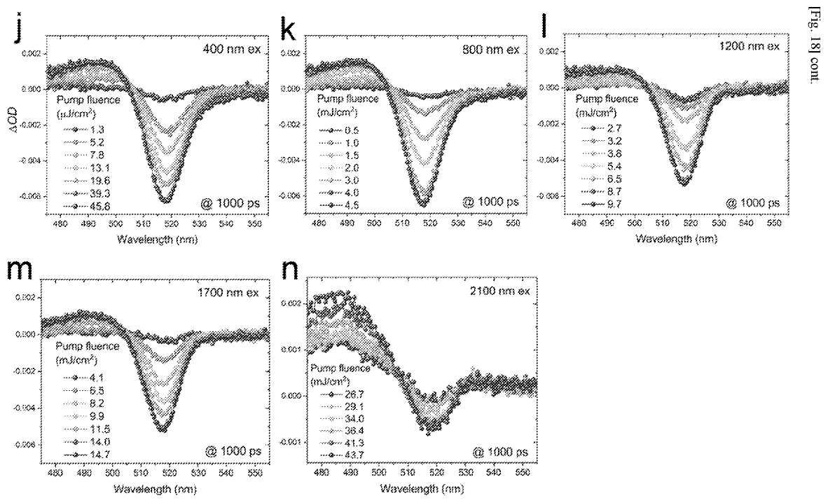

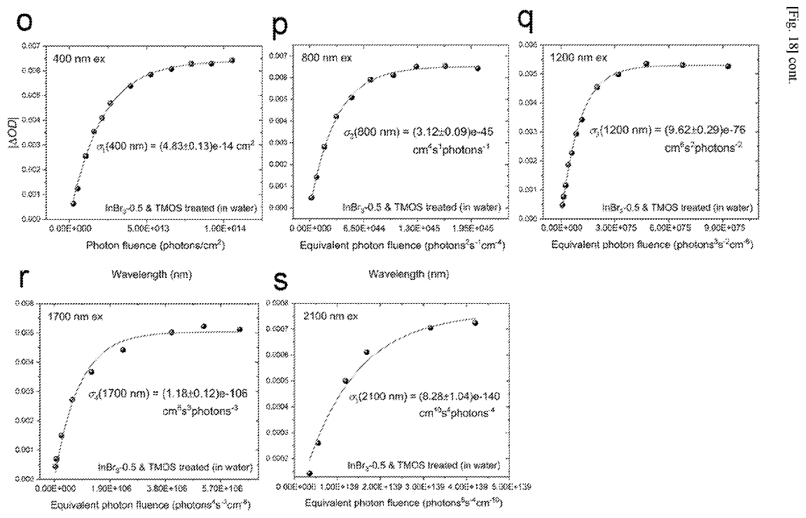

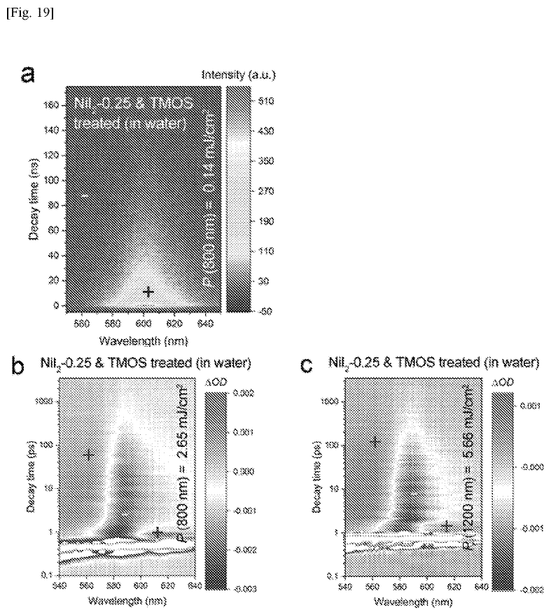

Combined with the absorbance spectrum of the reference sample, the molar absorption coefficient at first exciton was calculated to be α (545 nm) approximately 1.8×10⁵ M⁻¹cm⁻¹ close to the value of approximately 1.0×10⁵ M⁻¹ cm⁻¹ provided by Evident Technologies Inc and the σ₂ (800 nm) of approximately 1.48×10⁴ GM was also consistent with previously known data. Likewise, the TA spectral bleaching signals of the pristine CsPbBr₃ NCs and aqueous HPNCs showed a saturation trend with increasing pump fluence under single-photon to multi-photon excitation, which could be highly fitted by Equation 1 to derive corresponding single-/multi-photon absorption cross-sections σₙ (FIG. 16m-v, FIG. 17h-o, FIG. 18j-s, and FIG. 19d-i).

FIG. 15a, FIG. 16a, FIG. 17a and FIG. 18a show the up-conversion PL spectra of the pristine CsPbBr₃ NCs in toluene and HPNCs treated with representative metal halides (YCl₃-1.0, InBr₃-0.5 and NiI₂-0.25) and TMOS, in water, with excitation wavelengths ranging from 800 nm to 2100 nm. The linear relationship between the PL intensity and pump fluence in logarithmic scale is shown in FIG. 15b and FIG. 16b, with slopes, which show the order of MPE, of about 2, 3, 4, and 5 corresponding to the excitation of 800 nm, 1200 nm, 1700 nm, and 2100 nm, respectively, validating the occurrence of MPE.

Upon multiphoton excitation, the steady-state/time-resolved emission band position and shape of the HPNCs were basically the same as those under single-photon excitation (FIG. 16d-g, FIG. 17b-d, FIG. 18b-e, and FIG. 19a). In addition to the common trend of PL lifetime shortening caused by a significant increase in pump fluence due to the increase in the multiphoton order, the PL lifetime of representative aqueous HPNCs was comparable to (for NiI₂-0.25) or even much-longer (for YCl₃-1.0 and InBr₃-0.5) than the pristine CsPbBr₃ NCs under the same MPE order and similar pump fluence (FIG. 15f-h and FIG. 16c). All these results indicated that both the low dimensional perovskite shells and silicon-oxygen polymerized structures afforded excellent passivation and protection, enabling these aqueous HPNCs to maintain high water stability while performing at high pumping fluence. In particular, the blue and green emitting HPNCs with YCl₃-1.0 and InBr₃-0.5 post-treatments, respectively, were stable to high-order MPE of up to 4 and 5 photon excitation in water.

Table 1 summarizes the multiphoton action cross-sections (that is, σₙ×η) of the full-colour emitting HPNC aqueous solutions, which were 4 to 5 orders of magnitude larger than that of the most advanced organic molecules in dimethyl sulfoxide, and 1 to 4 orders of magnitude larger than that of aqueous solutions of inorganic semiconductor quantum dots (QDs) of elements in Groups II-VI of the Periodic Table of Elements, indicating that the nanocrystals disclosed herein may be a highly promising fluorophore labelling system for multi-photon microscopy applications.

TABLE 1
Properties of different potential materials for multiphoton excitation

| | | λₑₘ^{a} | η^{b} | σ₁ | σ₂ | σ₃ (×10⁻⁷⁶ | σ₄ (×10⁻¹⁰⁶ | σ₅ (×10⁻¹⁴⁰ | |
|---|---|---|---|---|---|---|---|---|---|
| Sample | Materials | (nm) | (%) | (×10⁻¹⁴ cm²) | (×10⁵ GM) | cm⁶s²photon⁻²) | cm⁸s³photon⁻³) | cm¹⁰s⁴photon⁻⁴) | Method ^{c} |
| Inventive | CsPbBr₃ NCs | 519 | ~32.6 | ~5.45 | ~2.76 | ~7.51 | ~1.2 | ~68.9 (2100 nm) | TA |
| Sample 1 | in toluene | | | (400 nm) | (800 nm) | (1200 nm) | (1700 nm) | | saturation |
| | | | | | | | | | (direct) |
| Inventive | InBr₃-0.5 & | 519 | ~80 | ~4.83 | ~3.12 | ~9.62 | ~1.18 | ~8.28 (2100 nm) | TA |
| Sample 2 | TMOS treated | | | (400 nm) | (800 nm) | (1200 nm) | (1700 nm) | | saturation |
| | HPNCs in water | | | | | | | | (direct) |
| Inventive | YCl₃-1.0 & | 458 | ~35 | ~9.78 | ~1.68 | ~5.8 | ~0.25 | N.A. | TA |
| Sample 3 | TMOS treated | | | (400 nm) | (800 nm) | (1200 nm) | (1700 nm) | | saturation |
| | HPNCs in water | | | | | | | | (direct) |
| Inventive | NiI₂-0.25 & | 600 | ~30 | ~8.05 | ~7.74 | ~15.2 | N.A. | N.A | TA |
| Sample 4 | TMOS treated | | | (400 nm) | (800 nm) | (1200 nm) | | | saturation |
| | HPNCs in water | | | | | | | | (direct) |
| Comparative | Organic | 503 | ~4.4 | N.A. | N.A. | 3.67 × 10⁻⁴ | N.A. | 1.92 × 10³ | SIDT^{d} |
| Sample 1 | molecule | | | | | (1197 nm) | | (2100 nm) | (direct) |
| | IPPS in DMSO | | | | | | | | |
| Comparative | CdSe/ZnS NCs | 550-605 | 35-71 | N.A. | 0.035-0.67 | N.A. | N.A. | N.A. | PL |
| Sample 2 | in water | | | | (700-1000 nm) | | | | comparison |
| | | | | | | | | | (indirect) |
| Comparative | ZnSe/ZnS NCs | 498-590 | 1.5-26 | N.A. | 1.4 × 10⁻⁴-0.014 | 0.03-0.4 | N.A. | N.A. | OAZ scans |
| Sample 3 | and their Cu/Mn | | | | (700-800 nm) | (700-800 nm) | | | (direct) |
| | doped series in | | | | | | | | |
| | water | | | | | | | | |
| Comparative | ZnS NCs in | 440 | N.A. | N.A. | ~0.2 | 0.027 | N.A. | N.A | OAZ scans ^{e} |
| Sample 4 | aqueous solution | | | | (532 nm) | (780 nm) | | | (direct) |
| Comparative | MAPbBr₃ NCs | ~520 | ~84 | N.A. | 4.8-62 | 39-330 | 4.2-360 | 460-2.9 × 10⁴ | OAZ scans |
| Sample 5 | in toluene | | | | (675-1000 nm) | (1050-1500 nm) | (1550-2000 nm) | (2050-2300 nm) | (direct) + PL |
| | | | | | | | | | comparison |
| | | | | | | | | | (indirect) |
| Comparative | CsPbBr₃ NCs | ~520 | ~55 | N.A. | 18-240 | 70-1.4 × 10³ | 13-1.3 × 10³ | 1.7 × 10³-1.2 × 10⁵ | OAZ scans |
| Sample 6 | in toluene | | | | (675-1000 nm) | (1050-1500 nm) | (1550-2000 nm) | (2050-2300 nm) | (direct) + PL |
| | | | | | | | | | comparison |
| | | | | | | | | | (indirect) |
| Comparative | MAPbBr₃/(OA)₂ | ~520 | ~92 | N.A. | 33-400 | 270-2.4 × 10³ | 23-2.6 × 10³ | 3.1 × 10³-2.2 × 10⁵ | OAZ scans |
| Sample 7 | PbBr₄ NCs in | | | | (675-1000 nm) | (1050-1500 nm) | (1550-2000 nm) | (2050-2300 nm) | (direct) + PL |
| | toluene | | | | | | | | comparison |
| | | | | | | | | | (indirect) |

^{a}λₑₘ refers to the emission centre wavelength of the material.
^{b}η refers to the PLQY.
^{c} Method refers to the method for single-/multi-photon absorption cross-section measurement.
^{d}SIDT refers to the static intensity-dependent transmission measurement.
^{e} OAZ scans refers to the open-aperture Z scan measurement.

### Example 7: In-Vivo Multiphoton Imaging

As a proof-of-concept (POC) of the potential of the aqueous HPNCs for multi-photon in-vivo imaging. Caenorhabditis elegans (C. elegans) ingested with aqueous HPNCs treated with InBr₃-0.5 and TMOS were imaged. The worms were first starved for 24 hours prior to exposure to the HPNCs, as starved C. elegans are known to feed quickly and indiscriminately on small particles. Upon 488 nm continuous light excitation, the ingested HPNCs were detected inside the pharynx of the worms after being exposed to HPNCs for about 10 minutes. In a video that was taken, it was observed that these C. elegans moved with a whip-like motion (or sinusoidal locomotion) on an agar plate, indicating that C. elegans readily fed on the HPNCs on the nematode growth medium (NGM) agar plates. This also validated successful operation of real-time in-vivo fluorescence imaging.

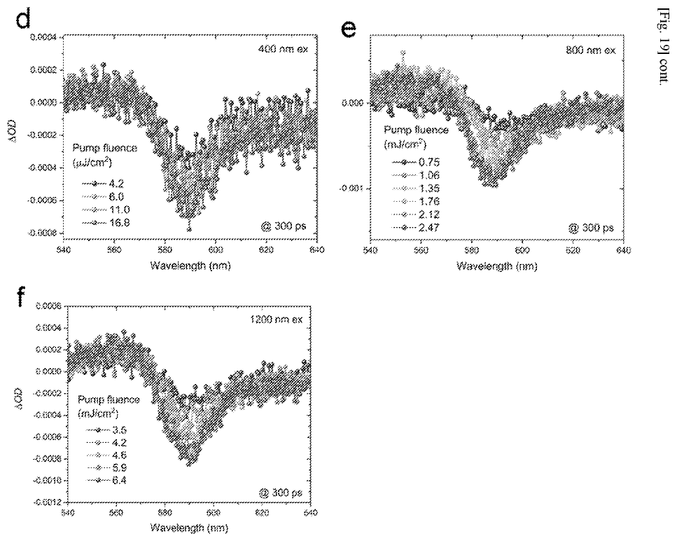

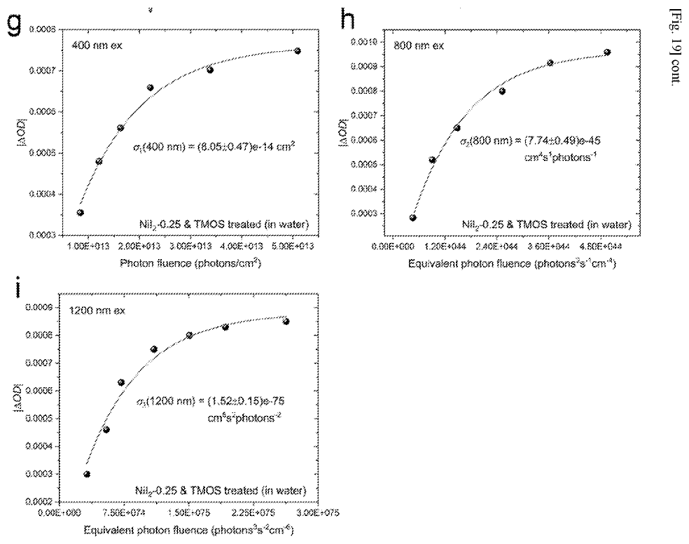

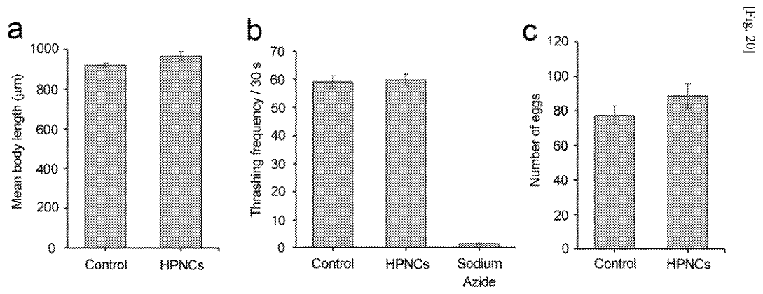

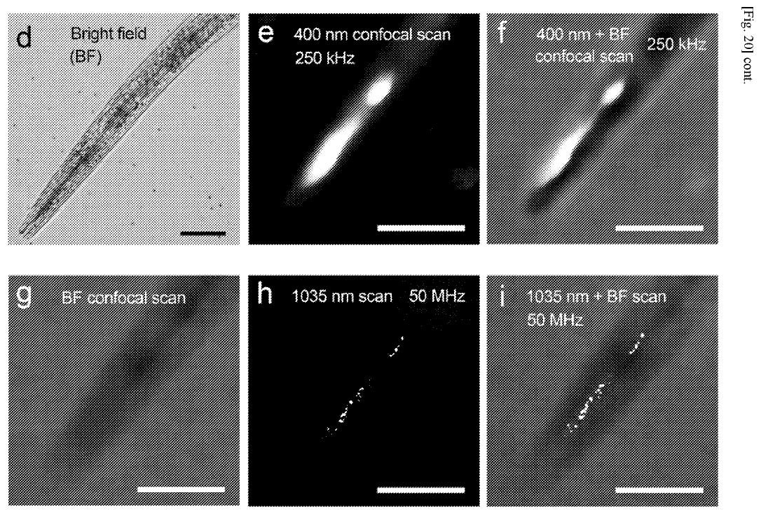

To ascertain whether these HPNCs were harmful to living organisms, the body length, locomotion, and reproductive rate of C. elegans that ingested HPNCs were studied. It was found that the body length of C. elegans that ingested the HPNCs was comparable with the control sample (FIG. 20a), with basically no adverse effect on locomotion when body thrash frequencies were measured (FIG. 20b). Moreover, the HPNCs did not affect the reproductive rates of C. elegans (FIG. 20c). These biological experiments indicated that the synthesized aqueous HPNCs possessed superior properties of low biological toxicity, which was attributed to the co-operative contributions of the low-lead perovskite shell, the doping/substitution of low-toxic metal ions, and the silicon-oxygen polymerized structure.

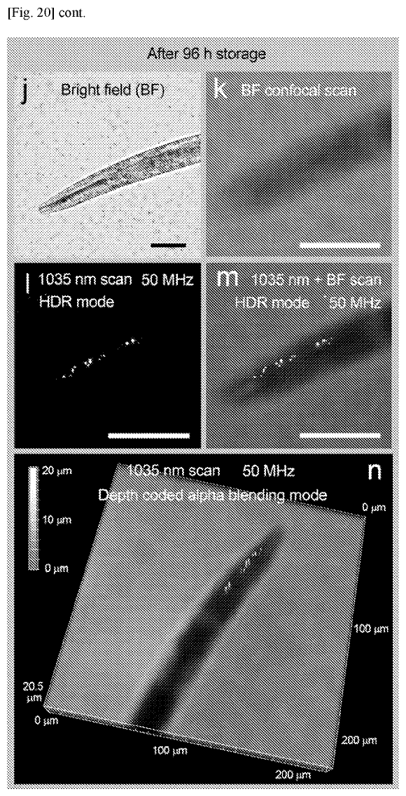

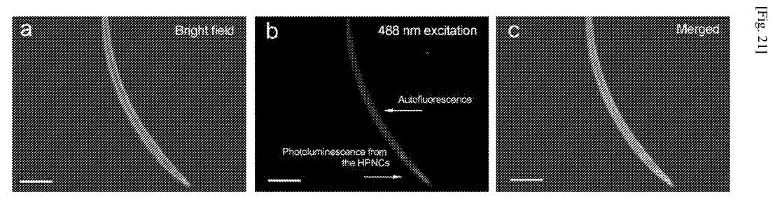

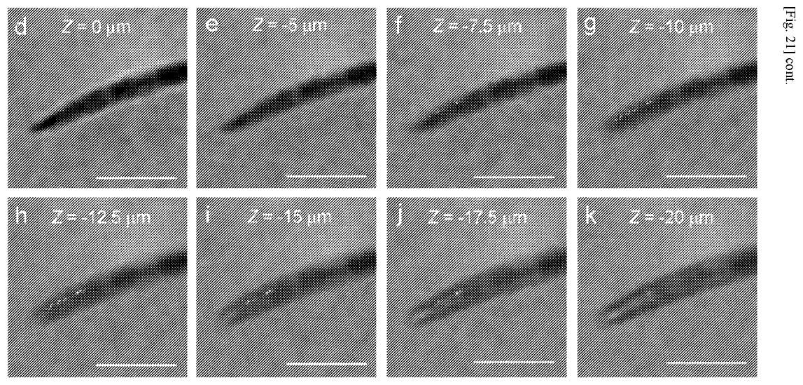

A further investigation of C. elegans, which had initially ingested HPNCs for about 10 minutes. found that the autofluorescence of the organisms was severe under continuous light-excitation (FIG. 21a-c) for down-conversion fluorescence imaging; while the spatial resolution was poor (only 2 to 3 μm) upon low-repetition, for example at 250 kHz, pulsed light excitation, even in a confocal microscopy system (FIG. 20d-f). In contrast, with multi-photon excitation, HPNCs in the pharynx of C. elegans could be clearly observed to be distributed along the oesophagus with high spatial resolution of approximately 90 nm under excitation of a near-infrared (NIR) femtosecond laser at high-repetition frequency (FIG. 20g-i). The NIR excitation wavelength was 1035 nm, half of which was exactly in the centre of the emission band of HPNCs treated with InBr₃-0.5 and TMOS, indicating that two 1035 nm photons were insufficient to excite the material, thus implying that the imaging proceeded via a three-photon excitation.

Furthermore, after being stored at 4° C. for 96 hours, the sample of C. elegans that ingested HPNCs still performed remarkably with high-quality MPE imaging in the high dynamic range (HDR) mode without observable PL quenching (FIG. 20j-m), despite completing a long-term continuous depth scan and 3D imaging under MPE (FIG. 20n, FIG. 21d-k). A depth scan video clearly showed that the HPNCs in the pharynx of C. elegans were distributed along the oesophagus with high spatial resolution. As the depth was changed, these bright regions come into focus and then defocused with the scan through the C. elegans body. These POC demonstrations validated that the high performance aqueous HPNCs were stable, non-bleaching and had low toxicity, which made them ideal candidates as a next-generation multi-photon fluorophores and in biological labelling.

### Industrial Applicability

The nanocrystal as defined above may be useful in full-colour displays, lasers, bioimaging, optoelectronics, spintronic devices, solar cells, memristors or radiation detectors.

The method of combining halide salt post-treatment and silica encapsulation may also be extended to other perovskite systems, including organic-inorganic hybrid perovskites, two-dimensional layered perovskites, and double perovskites.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A nanocrystal having a core-shell structure, wherein the core of the core-shell structure is at least partially encapsulated by the shell of the core-shell structure, wherein:
the core of the core-shell structure comprises a core perovskite structure having a formula ABX₃, wherein:
A is selected from the group consisting of at least one ion of one or more group 1 elements of the Periodic Table of Elements, an organic cation having a structure of R¹—(NHₓ)_{y}⁺ wherein R¹ is CH or alkyl, x is 2 or 3 and y is 1 or 2, as valency allows, and any mixture thereof;
B is at least one ion of one or more group 14 elements of the Periodic Table of Elements; and
X is a halide ion or any mixture thereof, and
the shell of the core-shell structure comprises a shell perovskite structure and a compound comprising silicon and oxygen, wherein the shell perovskite structure is different from the core perovskite structure and comprises a low-dimensional perovskite structure that is doped with a metal halide comprising a monovalent, divalent or trivalent metal ion.

2. The nanocrystal according to claim 1, wherein A is selected from the group consisting of CH₃NH₃⁺, CH(NH₂)₂⁺, Cs⁺, Rb⁺ and any mixture thereof, B is selected from the group consisting of Pb²⁺, Sn²⁺, Ge²⁺ and any mixture thereof, and X is selected from the group consisting of I⁻, Br⁻, Cl⁻, F⁻ and any mixture thereof.

3. The nanocrystal according to claim 1, wherein the monovalent, divalent or trivalent metal ion is selected from the group consisting of Na⁺, K⁺, Rb⁺, Ca²⁺, Sc³⁺, Cu⁺, Ga³⁺, Cd²⁺, Sn²⁺, Mn²⁺, Y³⁺, Zn²⁺, In³⁺, Ni²⁺, Co²⁺, Al³⁺, Mg²⁺, Fe²⁺, Fe³⁺, Pb²⁺, Bi³⁺, La³⁺, Ce³⁺, Pr³⁺, Nd³⁺, Pm³⁺, Sm³⁺, Eu³⁺, Gd³⁺, Tb³⁺, Dy³⁺, Ho³⁺, Er³⁺, Tm³⁺, Yb³⁺, Lu³⁺ and any mixture thereof.

4. The nanocrystal according to claim 1, wherein the low-dimensional perovskite shell further comprises a halide ion X′⁻ selected from the group consisting of I⁻, Br⁻, Cl⁻, For any mixture thereof.

5. The nanocrystal according to claim 1, wherein the low-dimensional perovskite structure in the shell perovskite structure is selected from the group consisting of a zero-dimensional perovskite structure, one-dimensional perovskite structure, two-dimensional perovskite structure and any mixture thereof.

6. The nanocrystal according to claim 1, wherein the core perovskite structure comprises three-dimensional γ-CsPbX₃, or the low-dimensional perovskite structure comprises a structure selected from the group consisting of zero-dimensional CsPbX₃ magic sized clusters, zero-dimensional Cs₄PbX₆, one-dimensional δ-CsPbX₃, two-dimensional CsPbX₃ nanoplatelets, two-dimensional CsPb₂X₅ and any mixture thereof.

7. (canceled)

8. The nanocrystal according to claim 1, wherein the compound comprising silicon and oxygen is selected from the group consisting of silica, silicate, an oligomeric silicon-oxygen compound, siloxane and any mixture thereof.

9. The nanocrystal according to claim 1, wherein the nanocrystal has a particle size in the range of about 40 nm to about 80 nm.

10. A process of preparing the nanocrystal according to claim 1, comprising a step of simultaneously mixing in a mixing solvent, a core perovskite structure having a formula ABX₃, a metal halide comprising a monovalent, divalent or trivalent metal ion and a precursor compound comprising silicon and oxygen, wherein:
A is selected from the group consisting of at least one ion of one or more group 1 elements of the Periodic Table of Elements, an organic cation having a structure of R¹—(NHₓ)_{y}⁺ wherein R¹ is CH or alkyl, x is 2 or 3 and y is 1 or 2, as valency allows, and any mixture thereof;
B is at least one ion of one or more group 14 elements of the Periodic Table of Elements; and
X is a halide ion.

11. The process according to claim 10, wherein prior to the mixing step, the process further comprises the step of dissolving the metal halide in a polar solvent comprising an alcohol, a fatty acid, a fatty amine, and an amine having a structure N(R²)₃, wherein R² is independently hydrogen or alkyl.

12. The process according to claim 11, wherein the polar solvent comprises about 50% to about 80% by volume of the alcohol, about 15% to about 25% by volume of the fatty acid, about 5% to about 15% by volume of the fatty amine, and about 1% to about 3% by volume of the amine having the structure N(R²)₃, wherein the total volume adds to 100%.

13. The process according to claim 10, wherein the polar solvent comprises oleic acid, oleylamine, ammonia and an alcohol selected from the group consisting of methanol, ethanol, isopropanol and any mixture thereof.

14. The process according to claim 10, wherein the metal halide is selected from the group consisting of NaX′, KX′, RbX′, CaX′₂, ScX′₃, CuX′, GaX′₃, CdX′₂, SnX′₂, MnX′₂, YX′₃, ZnX′₂, InX′₃, NiX′₂, CoX′₂, AlX′₃, MgX′₂, FeX′₂, FeX′₃, PbX′₂, BiX′₃, LaX′₃, CeX′₃, PrX′₃, NdX′₃, PmX′₃, SmX′₃, EuX′₃, GdX′₃, TbX′₃, DyX′₃, HoX′₃, ErX′₃, TmX′₃, YbX′₃, LuX′₃ and any mixture thereof, wherein X′ is independently selected from Cl, Br, I or F.

15. The process according to claim 10, wherein the metal halide is present at a concentration in the range of about 0.1 mM to about 400 mM.

16. The process according to claim 10, wherein the precursor compound comprising silicon and oxygen is selected from the group consisting of tetramethylorthosilicate (TMOS), tetraethylorthosilicate (TEOS), tetraisopropyl orthosilicate (TIPOS), (3-aminopropyl)triethoxysilane (APTES) and any mixture thereof.

17. The process according to claim 16, wherein the precursor compound comprising silicon and oxygen is present at a concentration in the range of about 2 mM to about 10 mM

18. The process according to claim 10, wherein the core perovskite structure is present at a concentration in the range of about 25 nM to about 70 nM.

19. The process according to claim 10, wherein the mixing solvent comprises a solvent selected from the group consisting of an alcohol, a fatty acid, a fatty amine, n-hexane, toluene, dichloromethane, an amine having a structure N(R²)₃ wherein R² is independently hydrogen or alkyl, and any mixture thereof.

20. The process according to claim 10, wherein the mixing step is performed at a temperature in the range of about 25° C. to about 35° C. for a duration in the range of about 1 hour to about 36 hours.

21. A substrate comprising the nanocrystal according to 1, wherein the substrate is selected from the group consisting of an aqueous solution, film, microcrystal, or bulk single crystal.

22. (canceled)
